(19) United States
(12) Patent Application Publication
(10) Pub. No.: US 2018/0342311 A1
(43) Pub. Date: Nov. 29, 2018

(54) INFORMATION PROCESSING SERVER, INFORMATION PROCESSING METHOD, AND INFORMATION PROCESSING SYSTEM

(71) Applicant: FUJITSU LIMITED, Kawasaki-shi (JP)

(72) Inventors: Yuta Watanabe, Kawasaki (JP); Akira Karasudani, Yamato (JP); Satoshi IWATA, Ebina (JP)

(73) Assignee: FUJITSU LIMITED, Kawasaki-shi (JP)

(21) Appl. No.: 15/988,398

(22) Filed: May 24, 2018

(30) Foreign Application Priority Data
May 29, 2017 (JP) 2017-105882

(51) Int. Cl. G16H 10/60; G16H 40/20; G06F 21/35

(52) U.S. Cl. CPC G16H 10/60; G06F 21/35; G16H 40/20

(57) ABSTRACT

An information processing server includes, a reception circuit configured to transmit a notification that a first measurable condition is satisfied in a case where an executor existence signal indicating existence of a work executor is detected, a notification that a second measurable condition is satisfied in a case where a subject existence signal indicating existence of a work subject is detected, and a notification that a third measurable condition is satisfied in a case where an access state of the work subject to information is detected, and a measurement circuit, coupled to the reception circuit, configured to start measurement of service time that is provided to the work subject when all measurable conditions from the first measurable condition to the third measurable condition are satisfied, and end the measurement of service time when any one of the measurable conditions is no longer satisfied.

### CROSS-REFERENCE TO RELATED APPLICATION

This application is based upon and claims the benefit of priority of the prior Japanese Patent Application No. 2017-105882, filed on May 29, 2017, the entire contents of which are incorporated herein by reference.

### FIELD

The embodiments discussed herein are related to an information processing server, an information processing method, and an information processing system.

### BACKGROUND

Accurately grasping waiting time until a service such as a medical treatment or receipt and payment is provided or time during which the service is provided in an outpatient medical treatment of a hospital or at a counter of a bank may help to improve efficiency of work or to improve a service.

One example of improving the service is to suggest waiting time until a hospital or bank administrator provides a service to a patient who came for medical treatment or a customer who came to a counter of a bank. Thereby, the patient or the customer easily and effectively uses the waiting time without waiting near a place where the service is provided.

From such a thing, for example, a nursing information management system capable of managing nursing information is proposed so as to evaluate temporal factors such as nursing time in a hospital.

Related technology is disclosed in Japanese Laid-open Patent Publication No. 2002-230169.

### SUMMARY

According to an aspect of the embodiments, an information processing server includes, a reception circuit configured to transmit a notification that a first measurable condition is satisfied in a case where an executor existence signal indicating existence of a work executor is detected, a notification that a second measurable condition is satisfied in a case where a subject existence signal indicating existence of a work subject is detected, and a notification that a third measurable condition is satisfied in a case where an access state of the work subject to information is detected, and a measurement circuit, coupled to the reception circuit, configured to start measurement of service time that is provided to the work subject when all measurable conditions from the first measurable condition to the third measurable condition are satisfied, and end the measurement of service time when any one of the measurable conditions is no longer satisfied.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating an example of a functional configuration of a management system as an information processing system;

FIG. 2 is an explanatory diagram illustrating an example of arrangement of each server and terminal in each room in the management system;

FIG. 3 is a diagram illustrating an example of a measurement result stored in a measurement result DB;

FIG. 4 is a diagram illustrating an example of information for managing each terminal stored in a memory;

FIG. 5 is an explanatory diagram illustrating an example of a hardware configuration of a management server;

FIG. 6 is a diagram illustrating an example of information that specifies a qualification ID stored in a qualification information DB and a doctor, a nurse, or the like associated with the qualification ID;

FIG. 7 is a sequence diagram illustrating an example of first measurable notification processing and first measurement end notification processing;

FIG. 8 is a sequence diagram illustrating an example of second measurable notification processing and second measurement end notification processing;

FIG. 9 is a sequence diagram illustrating an example of third measurable notification processing and third measurement end notification processing;

FIG. 10 is a sequence diagram illustrating an example of measurement start processing;

FIG. 11 is a sequence diagram illustrating an example of measurement end processing;

FIG. 12 is a flowchart illustrating an example of a flow of processing in which a beacon detection circuit monitors each beacon signal and transmits various notifications to a beacon reception circuit;

FIG. 13 is a flowchart illustrating an example of a flow of processing in which the beacon reception circuit transmits various notifications and IDs to a measurement circuit, based on various notifications received from the beacon detection circuit;

FIG. 14 is a flowchart illustrating an example of a flow of processing in which a qualification authentication circuit receives an authentication request from the beacon reception circuit and transmits various notifications to the beacon reception circuit;

FIG. 15 is a flowchart illustrating an example of a flow of processing in which a patient authentication circuit receives an authentication request from the beacon reception circuit and transmits various notifications to the beacon reception circuit;

FIG. 16 is a flowchart illustrating an example of a flow of processing in which a medical chart management circuit monitors whether or not an electronic medical chart is in an access state and transmits various notifications to the beacon reception circuit;

FIG. 17 is a flowchart illustrating an example of a flow of measurement start processing in which the measurement circuit starts measurement of medical treatment time;

FIG. 18 is a flowchart illustrating an example of a flow of measurement end processing in which the measurement circuit ends measurement of the medical treatment time;

FIG. 19 is a block diagram illustrating an example of a functional configuration of a management system according to a second embodiment;

FIG. 20 is a sequence diagram illustrating an example of second measurable notification processing and second measurement end notification processing according to the second embodiment;

FIG. 21 is a sequence diagram illustrating an example of a part of the measurement end processing according to the second embodiment;

FIG. 22 is a flowchart illustrating an example of a flow of processing in which the beacon reception circuit according to the second embodiment transmits various notifications and IDs to the measurement circuit, based on various notifications received from the beacon detection circuit;

FIG. 23 is a flowchart illustrating an example of a flow of the measurement end processing in which the measurement circuit ends the measurement of the medical treatment time; and

FIG. 24 is a block diagram illustrating an example of a functional configuration of a management system according to a third embodiment.

### DESCRIPTION OF EMBODIMENTS

However, a technology described in Japanese Laid-open Patent Publication No. 2002-230169 measures the time when a care subject and a caregiver approach each other, and thus, there is a problem in which there is a case where whether or not medical treatment is being performed may not be determined even if the care subject and the caregiver approach each other, and the time may not be accurately measured.

An information processing system according to the exemplary embodiment is a system that may more accurately measure service time. The service time includes, for example, the time when a doctor or a nurse performs medical treatment for a patient in a hospital, the time until a bank clerk receives a request item such as receipt and payment at a counter of a bank and a procedure of the receipt and payment is completed, and the like. Furthermore, the service time includes, for example, response time of a counter service in a government office.

The information processing system has a configuration or the like that includes, for example, a detection terminal, a terminal, and an information processing server.

The detection terminal transmits a notification that a first measurable condition is satisfied to the information processing server in a case where an executor existence signal indicating existence of a work executor who provides a service is detected within a predetermined region. The detection terminal may detect the executor existence signal by wire or wirelessly.

A transmission source of the executor existence signal includes, for example, a beacon, an IC tag, and the like which are carried by the work executor.

Detection of the executor existence signal may be detection performed by, for example, face authentication or the like of the work executor.

The work executor includes, for example, a doctor, a nurse, an inspection technician, or the like in a case of a hospital, a bank clerk or the like in a case of a bank, and a staff member or the like in a case of a government office, and the like.

In addition, the detection terminal transmits a notification that a second measurable condition is satisfied to the information processing server, in a case where a subject existence signal indicating existence of a work subject providing a service is detected in a predetermined region.

A transmission source of the subject existence signal includes, for example, a beacon carried by a work subject, an IC tag, and the like, in the same manner as the transmission source of the executor existence signal.

Detection of the subject existence signal may be detection performed by, for example, face authentication and the like of the work subject.

The work subject includes, for example, a patient and the like in a case of a hospital, a customer or the like in a case of a bank, a resident or the like in an administrative district of a government office in a case of the government office.

The predetermined region is a region where a doctor, a nurse, an inspection technician, or the like may physically treat a patient in a hospital, and includes, for example, a physical examination room, a treatment room, an X-ray room, and the like. In addition, the predetermined region includes, for example, a waiting room and the like near a counter in a bank, a government office or the like.

The terminal transmits a notification that a third measurable condition is satisfied to the information processing server, in a case where an access state of the work subject to information is detected.

The information of the work subject is, for example, an electronic medical chart in a case of a hospital, and includes receipt and payment data of an account in a case of a bank. The information of the work subject is displayed on a display or the like of the information processing server such as a personal computer.

The access state of the work subject to the information includes, for example, a state where the terminal accesses the electronic medical chart in a case of a hospital, a state where the terminal reads a magnetic stripe of a passbook and accesses account information of a customer in a case of a bank, and the like. In addition, the access state of the work subject to the information includes, for example, a state where the terminal reads a bar code or an IC tag and accesses the information of the work subject, and the like.

The information processing server starts measuring service time from notifications received from the detection terminal and the terminal, when all measurable conditions from the first measurable condition to the third measurable condition are satisfied. In other words, the information processing server starts the measurement of the service time, when there is a work executor and a work subject in the predetermined region, the information of the work subject is in the access state, and there is a high probability that the work executor may provide the service to the work subject.

Thereafter, the information processing server ends the measurement of the service time when any one of the measurable conditions is no longer satisfied. In other words, the information processing server ends the measurement of the service time when the work executor or the work subject or any combination thereof does not exist in the predetermined region, or when the access state of the work subject to the information is released.

By doing so, the information processing system may more accurately measure the service time, and thus, a work executor who provides a service may suggest more accurate waiting time to the work subject. Thereby, by confirming the waiting time suggested by a management system, the work subject may easily and effectively use the waiting time without waiting near a place where the service is provided.

In addition, by detecting the beacon signals of the work executor and the work subject and the access state of the work subject to the information, the information processing system may perform automatic measurement without performing work for the work executor or the like to measure the service time.

In addition, even if the terminal detects release of the access state of the work subject to the information and provides a notification that the third measurable condition is no longer satisfied, in a case where the detection terminal continuously detects the subject existence signal in the predetermined region, the information processing server may not end the measurement of the service time.

This considers, for example, in a case of an outpatient medical treatment of a hospital, a doctor physically examines a patient in a physical examination room, then, a nurse treats the patient in a treatment room, and the doctor physically treats the patient again in the physical examination room. In this case, if the doctor ends display of the electronic medical chart in the physical examination room at the end of ending the first physical examination, the measurement of the service time is ended regardless of a subsequent treatment or physical treatment. Accordingly, even if the terminal detects end of the display of the electronic medical chart and provides a notification that the third measurable condition is no longer satisfied, in a case where the detection terminal continuously detects the patient beacon signal in the physical examination room or the treatment room, the information processing server does not end the measurement of the service time.

Hereinafter, one embodiment of the exemplary embodiment is described, but the exemplary embodiment is not limited to this embodiment at all.

### First Embodiment

In a first embodiment, a management system 10 according to the present embodiment as an information processing system is introduced to a hospital and more accurately measures medical treatment time which is service time of the hospital. Thereby, the hospital may suggest a more accurate waiting time to a patient, based on a measurement result, and thus, the patient may effectively utilize waiting time without waiting near a physical examination room.

Here, the medical treatment time is the sum of the physical examination time and the medical treatment time in the present embodiment.

In the present embodiment, the physical examination time is time when a doctor or the like mainly investigates a body of a patient. In addition, in the present embodiment, the medical treatment time is time when a nurse or the like mainly treats illness or injury to cure.

Management System (Information Processing System)

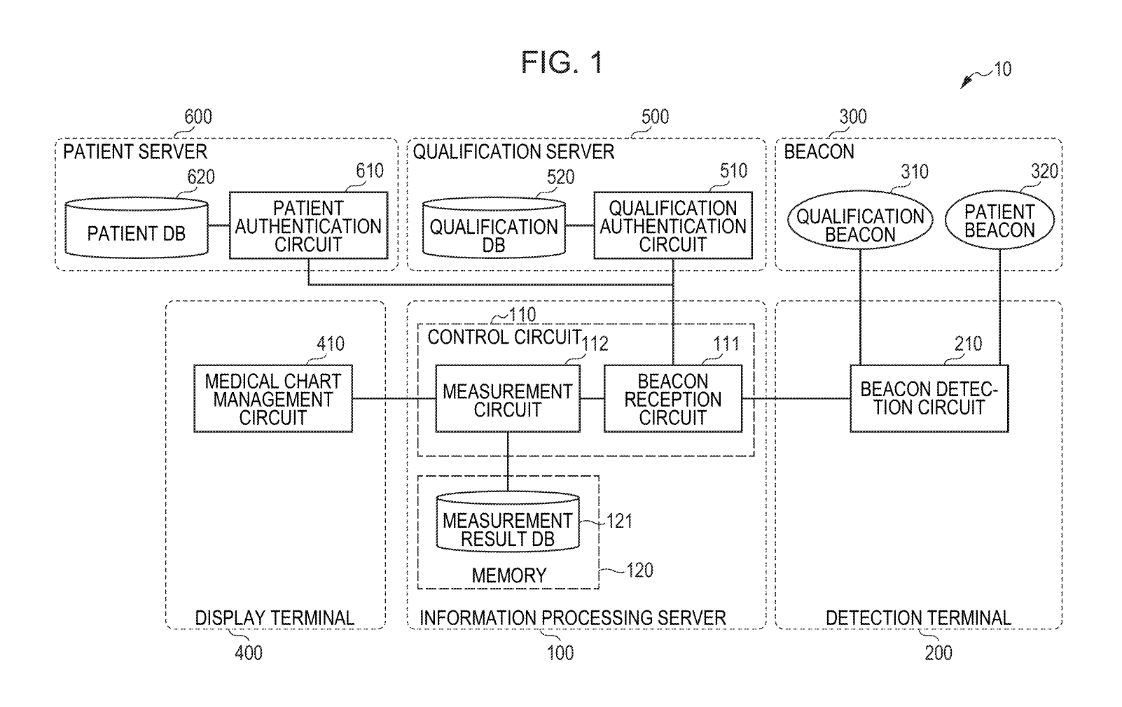

FIG. 1 is a block diagram illustrating an example of a functional configuration of a management system 10. The management system 10 as an information processing system includes a management server 100 as an information processing server, a detection terminal 200, a qualification beacon 310 and a patient beacon 320, a display terminal 400 as a terminal, a qualification server 500, and a patient server 600.

The management server 100 is communicably connected to the detection terminal 200, the display terminal 400, the qualification server 500, and the patient server 600.

The detection terminal 200 may receive and detect a qualification beacon signal (executor existence signal) transmitted from a qualification beacon 310 carried by a doctor, a nurse, or the like as a work executor, in a physical examination room, a treatment room, or the like it. In a case where a qualification beacon signal is detected, the detection terminal 200 transmits a notification that a first measurable condition is satisfied to the management server 100.

In addition, in the same manner as the qualification beacon signal, the detection terminal 200 may receive and detect a patient beacon signal (subject existence signal) transmitted from the patient beacon 320 carried by a patient as a work subject in a physical examination room or a treatment room. In a case where the patient beacon signal is detected, the detection terminal 200 transmits a notification that the second measurable condition is satisfied to the management server 100.

In a case where the fact that information of a patient such as an electronic medical chart is in an access state in a physical examination room or a medical treatment room is detected, the display terminal 400 transmits a notification that a third measurable condition is satisfied to the management server 100.

The management server 100 starts measurement of the medical treatment time as service time, when all measurable conditions from a first measurable condition to a third measurable condition are satisfied from a notification received from the detection terminal 200 and the display terminal 400. In other words, when a patient exists together with a doctor, a nurse, or the like in a physical examination room or a treatment room and an electronic medical chart of the patient is displayed, that is, when there is a high possibility that a patient may be medically treated by a doctor, a nurse, or the like, the management server 100 starts the measurement of the medical treatment time.

Thereafter, when any one of the measurable conditions is no longer satisfied, the management server 100 ends the measurement of the medical treatment time. In other words, when a work executor such as a doctor or a nurse or a work subject which is a patient or any combination thereof does not exist in the physical examination room or the treatment room, or when an electronic medical chart of the patient is in a non-access state, the management server 100 ends the measurement of the medical treatment time.

All the notifications that the first to third measurable conditions are satisfied may be referred to as “measurable notifications”. In addition, all the notifications that the first to third measurable conditions are no longer satisfied may be referred to as “measurement end notifications”.

The management server 100 transmits an authentication request to the qualification authentication circuit 510 included in the qualification server 500, based on a qualification ID included in a qualification beacon signal and specifies a doctor, a nurse, or the like carrying the qualification beacon 310.

The management server 100 transmits an authentication request to the patient authentication circuit 610 included in the patient server 600, based on a patient ID included in a patient beacon signal and specifies a patient carrying the patient beacon 320.

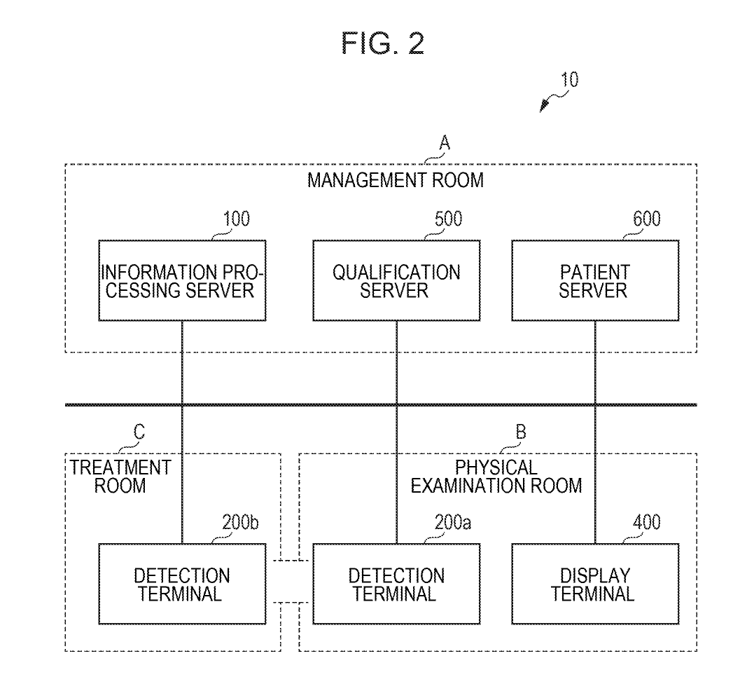

FIG. 2 is an explanatory diagram illustrating an example of arrangement of each device in the management system 10 in each room.

As illustrated in FIG. 2, a management room A is a room where the management server 100, the qualification server 500, and the patient server 600 are installed to be managed.

The physical examination room B is a room where a doctor or the like physically examines a patient. In the physical examination room B, a detection terminal 200a and the display terminal 400 are arranged. The detection terminal 200a may detect each beacon signal in the physical examination room B.

The treatment room C is a room where a nurse or the like treats a patient according to a result of the physical examination. In the treatment room C, for example, if a surgery is performed, a treatment such as wrapping a bandage around an affected part of the patient by a nurse is performed. A detection terminal 200b is disposed in the treatment room C. The detection terminal 200b may detect each beacon signal of the treatment room C.

It is assumed that, when a patient moves between the physical examination room B and the treatment room C, the patient beacon signal is continuously detected by the detection terminals 200a and 200b.

In addition, since configurations of the detection terminals 200a and 200b are the same as each other, the detection terminals are collectively referred to as a “detection terminal 200” hereinafter, unless distinguished from each other.

Next, returning to FIG. 1, functional configurations and hardware configurations of the respective devices are described.

Since hardware configurations of the management server 100, the display terminal 400, the qualification server 500, and the patient server 600 are the same as each other, the management server 100 is described and description of the hardware configurations of the other devices is omitted.

Functional Configuration of Management Server

The management server 100 includes a control circuit 110 and a memory 120.

Control Circuit

The control circuit 110 executes various programs stored in the memory 120 and controls the entire management server 100.

The control circuit 110 includes a beacon reception circuit 111 and a measurement circuit 112.

The beacon reception circuit 111 transmits a notification that the first measurable condition is satisfied to the measurement circuit 112 together with the qualification ID, based on a qualification beacon signal detection notification from the beacon detection circuit 210 of the detection terminal 200.

In addition, the beacon reception circuit 111 transmits a notification that the second measurable condition is satisfied to the measurement circuit 112 together with the patient ID, based on a patient beacon signal detection notification from the beacon detection circuit 210 of the detection terminal 200.

The beacon reception circuit 111 transmits a notification that the first measurable condition is no longer satisfied to the measurement circuit 112 together with the qualification ID, based on a qualification beacon signal loss notification from the beacon detection circuit 210 of the detection terminal 200.

The beacon reception circuit 111 transmits a notification that the second measurable condition is no longer satisfied to the measurement circuit 112 together with the patient ID, based on a patient beacon signal loss notification from the beacon detection circuit 210 of the detection terminal 200.

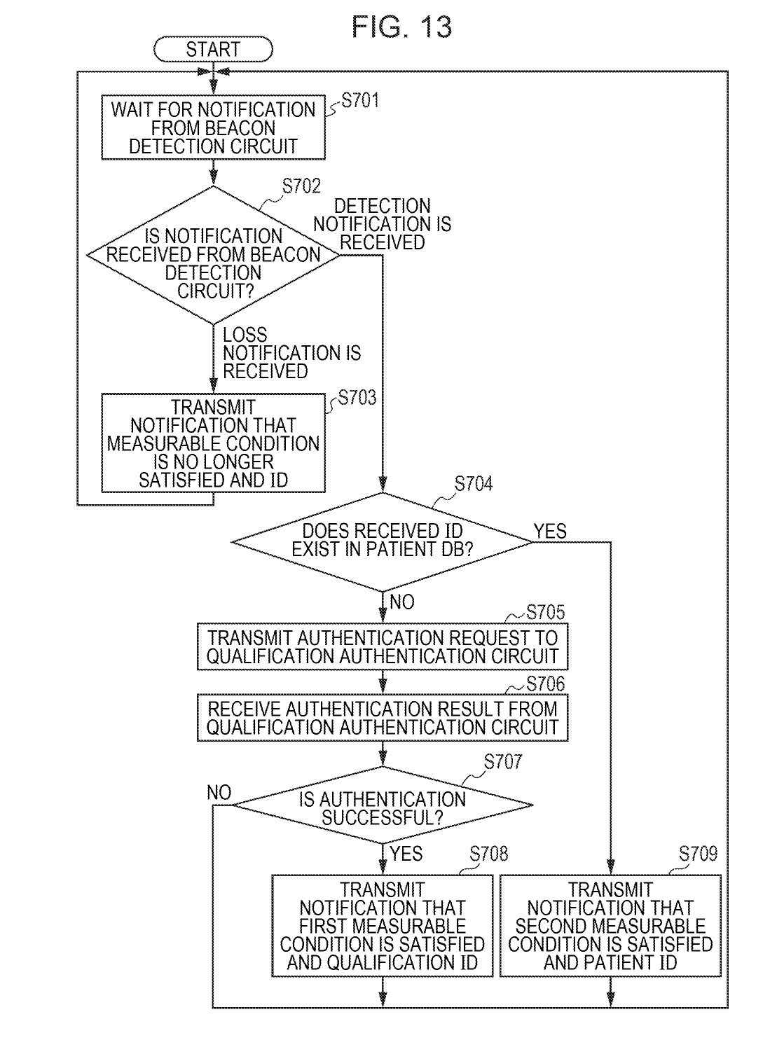

Specifically, the beacon reception circuit 111 waits for a notification from the beacon detection circuit 210 (see S701 in FIG. 13) and determines whether or not the notification is received from the beacon detection circuit 210 (see S702 in FIG. 13). If it is determined that a beacon signal loss notification is received from the beacon detection circuit 210, the beacon reception circuit 111 transmits a notification that the measurable condition is no longer satisfied and an ID (see S703 in FIG. 13).

If it is determined that a beacon signal detection notification is received from the beacon detection circuit 210, the beacon reception circuit 111 further determines whether or not the received ID exists in a patient information DB (see S704 in FIG. 13).

If it is determined that the received ID does not exist in the patient information DB, the beacon reception circuit 111 transmits an authentication request to the qualification authentication circuit 510 (see S705 in FIG. 13), and thereafter, receives an authentication result from the qualification authentication circuit 510 (See S706 in FIG. 13). When receiving the authentication result, the beacon reception circuit 111 determines whether or not the authentication result is successful in authentication (see S707 in FIG. 13). When determining that the authentication result fails in authentication, the beacon reception circuit 111 waits for notification from the beacon detection circuit 210. When determining that the authentication result is successful in authentication, the beacon reception circuit 111 adds a qualification ID to the notification that the first measurable condition is satisfied, transmits the notification and the qualification ID to the measurement circuit 112, and waits for a notification from the beacon detection circuit 210 (See S708 in FIG. 13).

When determining that the received ID exists in the patient information DB, the beacon reception circuit 111 adds the patient ID to the notification that the second measurable condition is satisfied and transmits the patient ID and the notification to the measurement circuit 112 (see S709 in FIG. 13).

Thereby, only it is possible to measure the time when a doctor or nurse actually faces a patient as the medical treatment time.

When receiving notifications that all the measurable conditions from the first measurable condition to the third measurable condition are satisfied are received from the beacon reception circuit 111 or the medical chart management device 410, the measurement circuit 112 performs measurement start processing of starting measurement of medical treatment time.

In addition, when receiving a notification that any one measurable condition is no longer satisfied is received from the beacon reception circuit 111 or the medical chart management device 410, the measurement circuit 112 performs a measurement end processing of ending the measurement of the medical treatment time.

Measurement Start Processing

Specifically, the measurement circuit 112 first waits for a notification from the beacon reception circuit 111 or the medical chart management device 410 as the measurement start processing (see S1101 in FIG. 17).

The measurement circuit 112 determines whether or not a notification is received from the beacon reception circuit 111 or the medical chart management device 410 (see S1102 in FIG. 17). If it is determined that one of the notifications that the first to third measurable conditions are no longer satisfied is received from the beacon reception circuit 111 or the medical chart management device 410, the measurement circuit 112 proceeds to measurement end processing which is described below.

If it is determined that the measurable notification is a notification that the first measurable condition is satisfied and a qualification ID (see S1103 in FIG. 17), the measurement circuit 112 which determines that the measurable notification is received saves the qualification ID in a measurement result DB 121 (see S1104 in FIG. 17).

If it is determined that the measurable notification is a notification that the second measurable condition is satisfied and a patient ID (see S1105 in FIG. 17), the measurement circuit 112 which determines that the measurable notification is received saves the patient ID in the measurement result DB 121 (see S1106 in FIG. 17).

If it is determined that the measurable notification is a notification that the third measurable condition is satisfied (see S1107 in FIG. 17), the measurement circuit 112 which determines that the measurable notification is received determines whether or not to the patient ID is added to the notification that the third measurable condition is satisfied (see S1108 in FIG. 17). When it is determined that the patient ID is added, the measurement circuit 112 saves the patient ID in the measurement result DB 121 (see S1109 in FIG. 17).

Next, the measurement circuit 112 determines whether or not notifications that all the measurable conditions from the first measurable condition to the third measurable condition are satisfied are received (see S1110 in FIG. 17). If it is determined that the notifications that all the measurable conditions from the first measurable condition to the third measurable condition are satisfied are received, the measurement circuit 112 starts measurement of medical treatment time (see S1111 in FIG. 17). If it is determined that the notifications that all the measurable conditions from the first measurable condition to the third measurable condition are satisfied are received, the measurement circuit 112 returns the processing to S1101.

Measurement End Processing

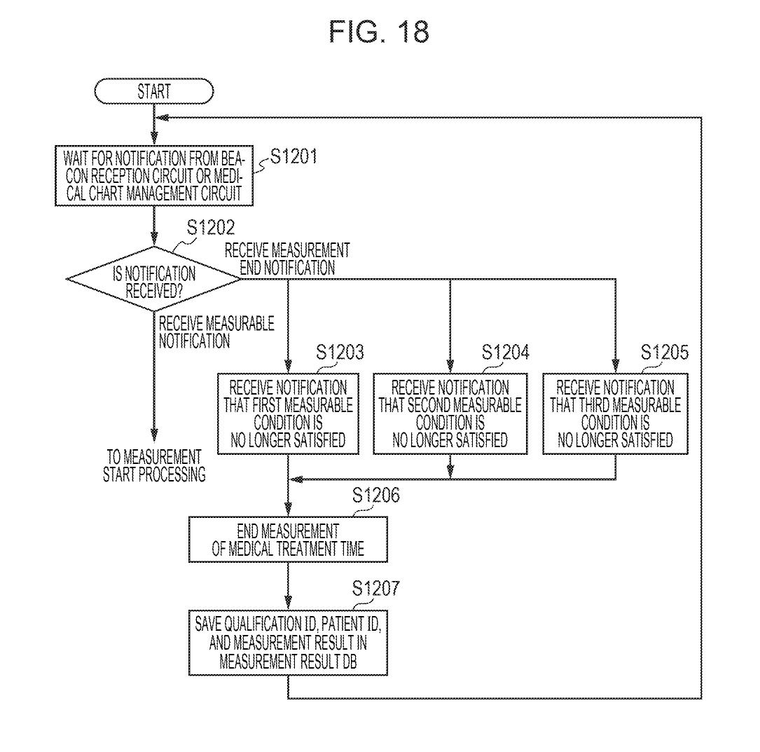

The measurement circuit 112 first waits for a notification from the beacon reception circuit 111 or the medical chart management device 410 as measurement end processing (see S1201 in FIG. 18). The measurement circuit 112 determines whether or not a notification is received from the beacon reception circuit 111 or the medical chart management device 410 (see S1202 in FIG. 18). If it is determined that the measurable notification is received, the measurement circuit 112 proceeds to the measurement start processing described above.

If it is determined that the measurement end notification is received, the measurement circuit 112 ends the measurement of the medical treatment time (see S1203 to S1206 in FIG. 18), and associates the qualification ID, the patient ID, and the measurement result to save in the measurement result DB 121 (see S1207 in FIG. 18).

The memory 120 includes the measurement result database 121. Hereinafter, a database may also be referred to as a “DB”.

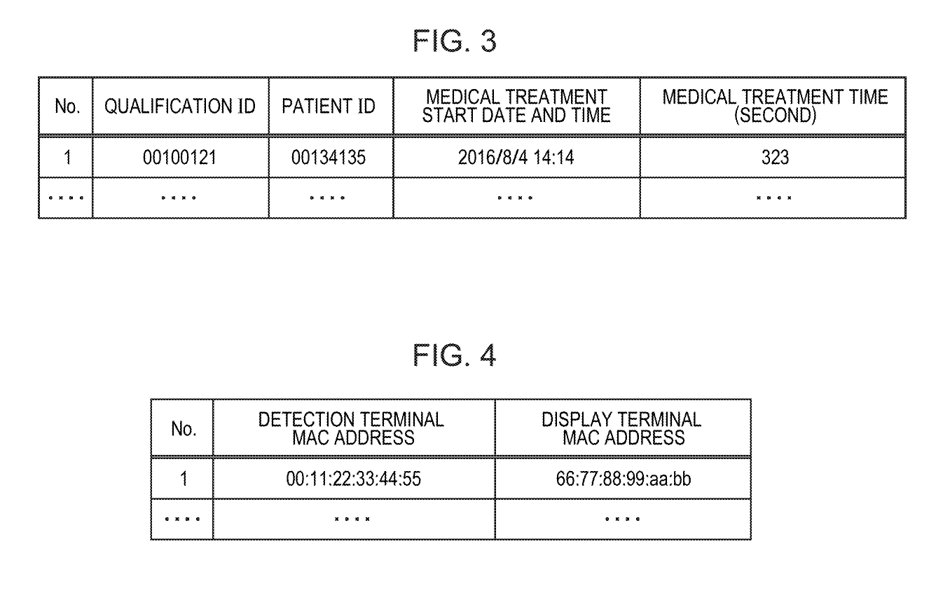

FIG. 3 is a diagram illustrating an example of measurement results stored in the measurement result DB 121.

As illustrated in FIG. 3, the measurement result DB 121 stores, data items of “a qualification ID, a patient ID, medical treatment start date and time, and medical treatment time (second)” in association with each other as measurement results.

The “qualification ID” is an identifier for uniquely specifying a work executor having a medicine-related qualification. The medicine-related qualification is used for, for example, a doctor, a nurse, an inspection technician, or the like.

The “patient ID” is an identifier for uniquely specifying a patient.

The “medical treatment start date and time” is information on the date and time when a medical treatment is started.

The “medical treatment time (second)” is information on the measured medical treatment time.

FIG. 4 is a diagram illustrating an example of information for managing each terminal stored in the memory 120.

As illustrated in FIG. 4, the memory 120 stores data items of “a detection terminal media access control (MAC) address, and a display terminal MAC address” in association with each other.

In the present embodiment, the “detection terminal MAC address” is a physical address uniquely assigned to each detection terminal on the network in principle.

In the present embodiment, the “display terminal MAC address” is a physical address uniquely assigned to each terminal on the network in principle.

Hardware Configuration of Management Server

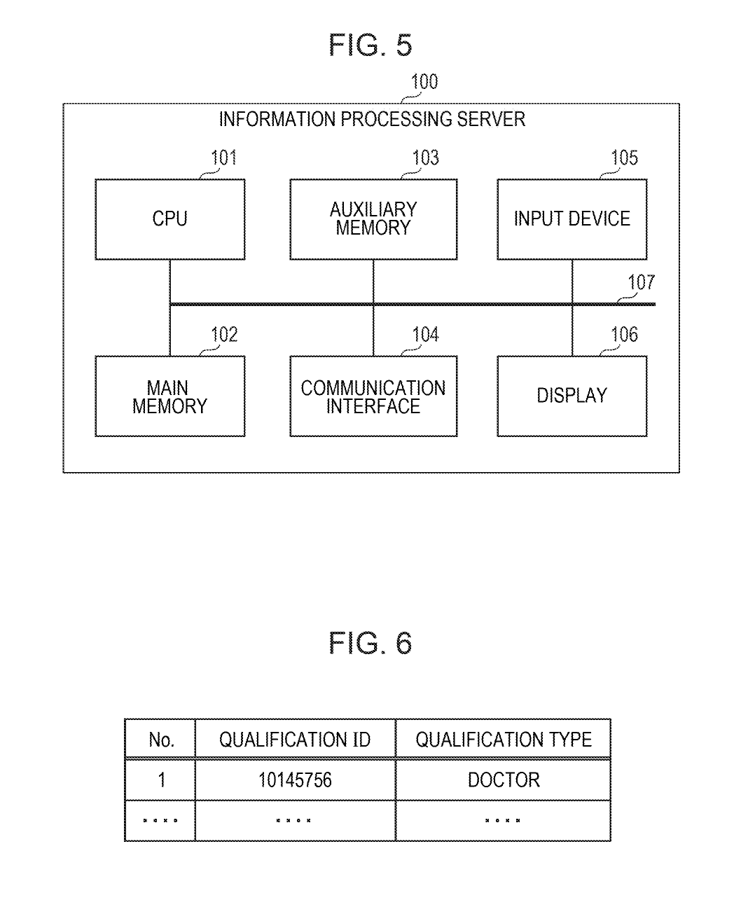

FIG. 5 is an explanatory diagram illustrating an example of a hardware configuration of the management server 100.

As illustrated in FIG. 5, the management server 100 includes the following respective circuits. The respective circuits are communicably connected to each other via a bus 107.

A central processing (CPU) 101 is a processing device that performs various controls and calculations. The CPU 101 realizes various functions by executing an operating system (OS) and a program stored in a main storage device 102 and the like.

In addition, the CPU 101 controls the entire operation of the management server 100. In the present embodiment, the CPU 101 controls the entire operation of the management server 100, but the exemplary embodiment is not limited to this, and may be, for example, a field programmable gate array (FPGA) or the like.

The main storage device 102 stores various programs and stores data and the like demanded for executing various programs.

The main storage device 102 includes a read only memory (ROM) and a random access memory (RAM) which are not illustrated.

The ROM stores various programs such as a basic input and output system (BIOS), and the like.

The RAM functions as a work range expanded when various programs stored in the ROM are executed by the CPU 101. The RAM is not limited in particular and may be appropriately selected depending on a purpose thereof. For example, a dynamic random access memory (DRAM), a static random access memory (SRAM), or the like may be used as the RAM.

An auxiliary storage device 103 is not limited in particular as long as various kinds of information are able to be stored, may be appropriately selected depending on a purpose thereof, and may be a solid state drive, a hard disk drive, or the like. In addition, the auxiliary storage device 103 may be a portable storage device such as a compact disc (CD) drive, a digital versatile disc (DVD) drive, or a Blu-ray (registered trademark) disc (BD) drive.

A communication interface 104 is not limited in particular, a publicly known device may be appropriately used as the communication interface, and for example, a communication device or the like which uses wireless communication or wire communication may be used for the communication interface.

An input device 105 is not limited in particular as long as various requests for the management server 100 is able to be received, a publicly known device may be appropriate used as the input device, and, for example, a keyboard, a mouse, a touch panel, and the like may be used for the input device.

A display 106 is not limited in particular, and a publicly known device may be appropriately used as the display, and, for example, a liquid crystal display, an organic EL display, or the like may be used for the display.

The management server 100 may be a part of a cloud that is a group of computers on a network.

Detection Terminal

Returning to FIG. 1, the detection terminal 200 includes a beacon detection circuit 210.

In a case where a qualification beacon signal is detected, the beacon detection circuit 210 notifies that a first measurable condition is satisfied. In addition, in a case where a patient beacon signal is detected, the beacon detection circuit notifies that a second measurable condition is satisfied.

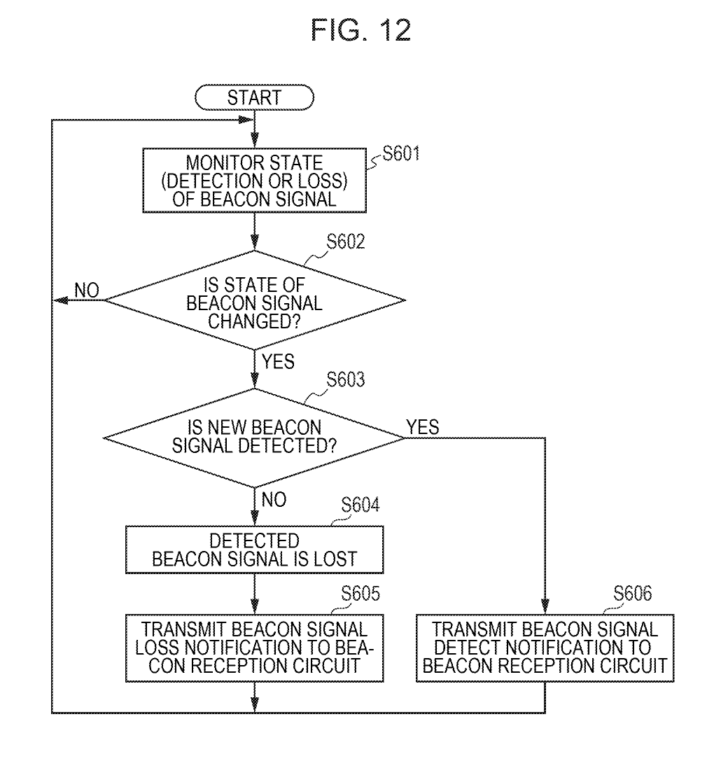

Specifically, the beacon detection circuit 210 monitors a state (detection or loss) of each beacon signal in the qualification beacon signal or a patient beacon signal (see S601 in FIG. 12), and determines whether or not the state of the beacon signal is changed (See S602 in FIG. 12). If it is determined that the state of the beacon signal is not changed, the beacon detection circuit 210 continuously monitors the beacon signal. If it is determined that the state of the beacon signal is changed, the beacon detection circuit 210 determines whether or not a new beacon signal is detected (see S603 in FIG. 12). If it is determined that the new beacon signal is not detected, the beacon detection circuit 210 determines that the detected beacon signal is lost (see S604 in FIG. 12), transmits a beacon signal loss notification to the beacon reception circuit 111 (see S605 in FIG. 12), and continuously monitors the beacon signal.

In addition, if it is determined that the new beacon signal is detected, the beacon detection circuit 210 transmits a beacon signal detection notification to the beacon reception circuit 111 (see S606 in FIG. 12) and continuously monitors the beacon signal.

The qualification beacon 310 is an executor identification device carried by a doctor, a nurse, an inspection technician, or the like as a work executor.

The qualification beacon 310 transmits a qualification beacon signal including a qualification ID which is specified by a work executor that performs execution.

The patient beacon 320 is a subject identification device carried by a patient as a work subject.

The patient beacon 320 transmits a patient beacon signal including a patient ID for that is specified by a work subject which performs execution.

Returning to FIG. 1, the qualification server 500 includes a qualification authentication circuit 510 and a qualification information DB 520.

The qualification authentication circuit 510 performs authentication, based on a qualification ID transmitted from the beacon reception circuit 111.

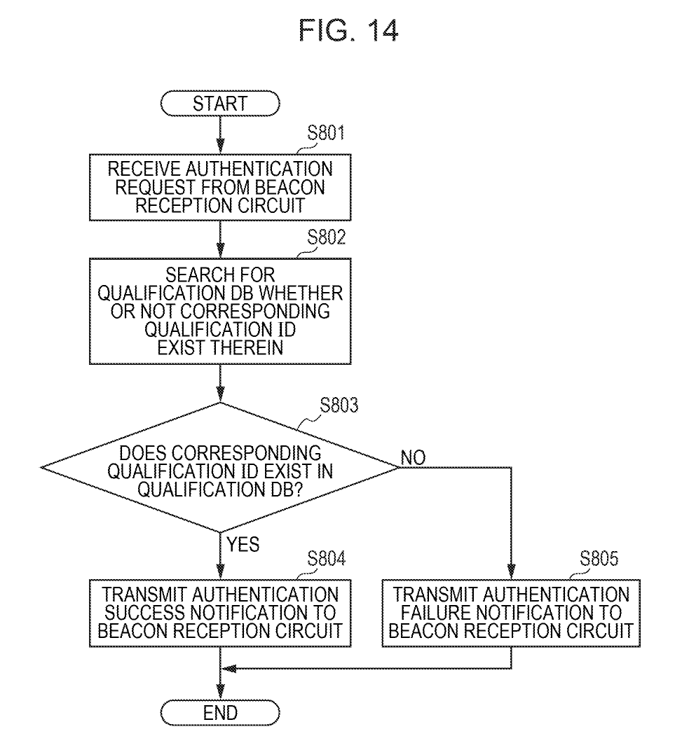

Specifically, the qualification authentication circuit 510 receives an authentication request based on the qualification ID from the beacon reception circuit 111 (see S801 in FIG. 14), and performs search processing, based on whether or not a corresponding qualification ID exists in the qualification information DB 520 (See S802 in FIG. 14).

The qualification authentication circuit 510 that performs the search processing determines whether or not the corresponding qualification ID exists in the qualification information DB 520 (see S803 in FIG. 14). If it is determined that the corresponding qualification ID exists in the qualification information DB 520 the qualification authentication circuit 510 transmits an authentication result notification to the effect that authentication is successful to the beacon reception circuit 111 (see S804 in FIG. 14). If it is determined that the corresponding qualification ID does not exist in the qualification information DB 520, the qualification authentication circuit 510 transmits an authentication result notification to the effect that the authentication fails to the beacon reception circuit 111 (see S805 in FIG. 14).

The qualification information DB 520 stores the qualification ID and information for specifying a doctor, a nurse, or the like associated with the qualification ID.

FIG. 6 is a diagram illustrating an example of information for specifying the qualification ID stored in the qualification information DB 520, and a doctor, a nurse, or the like associated with the qualification ID.

As illustrated in FIG. 6, the qualification information DB 520 stores data items of “the qualification ID and a qualification type” in association with each other.

As described above, the “qualification ID” is an identifier for uniquely specifying a work executor having a medicine-related qualification.

In the present embodiment, the “qualification type” is information indicating the type of medicine-related qualification such as a doctor, a nurse, a medical treatment technician, or the like.

As illustrated in FIG. 1, the patient server 600 includes a patient authentication circuit 610 and a patient information DB 620.

The patient authentication circuit 610 performs authentication, based on a patient ID transmitted from the beacon reception circuit 111.

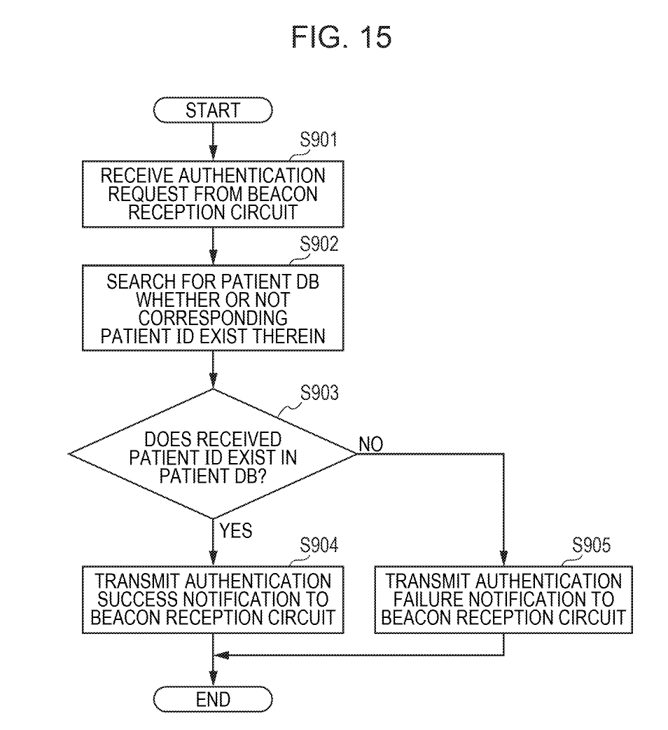

Specifically, the patient authentication circuit 610 receives an authentication request based on the patient ID from the beacon reception circuit 111 (see S901 in FIG. 15), and performs search processing, based on whether or not a corresponding patient ID exists in the patient information DB 620 (see S902 in FIG. 15).

The patient authentication circuit 610 that performs the search processing determines whether or not the corresponding patient ID exists in the patient information DB 620 (see S903 in FIG. 15). If it is determined that the corresponding patient ID exists in the patient information DB 620, the patient authentication circuit 610 transmits an authentication result notification to the effect that the authentication is successful to the beacon reception circuit 111 (see S904 in FIG. 15). If it is determined that the corresponding patient ID does not exist in the patient information DB 620, the patient authentication circuit 610 transmits an authentication result notification to the effect that the authentication fails to the beacon reception circuit 111 (see S905 in FIG. 15).

The patient information DB 620 stores the patient ID and patient information associated with the patient ID.

The patient information is, for example, personal information and the like on the patient. The personal information on the patient includes, for example, information such as a name, an age, a date of birth, an address, a telephone number, sex, an insurance card number, a past medical history, and the like.

Display Terminal

Returning to FIG. 1, the display terminal 400 includes a medical chart management device 410.

In a case where a display signal of information of a work subject is detected, the medical chart management device 410 transmits a notification that a third measurable condition is satisfied to the measurement circuit 112.

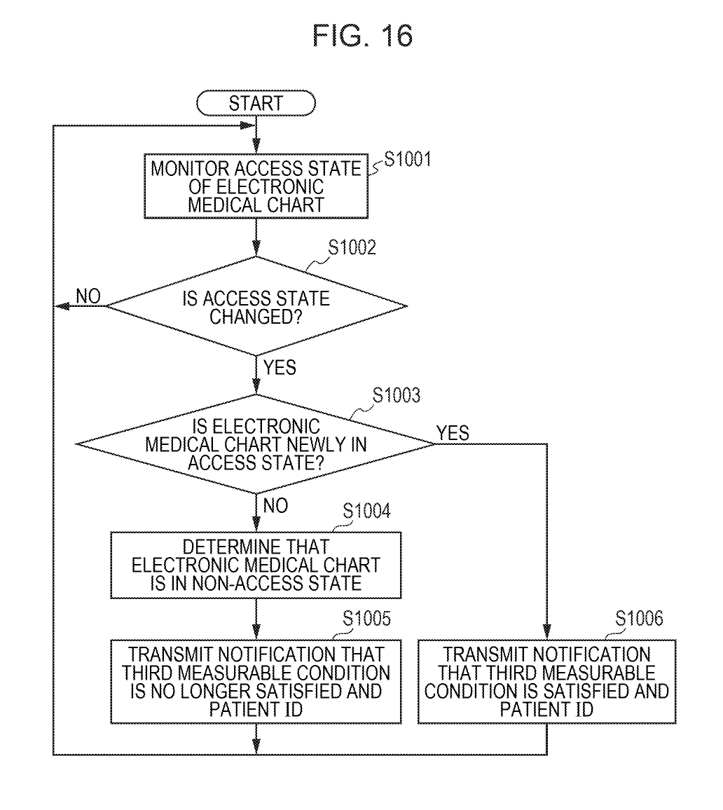

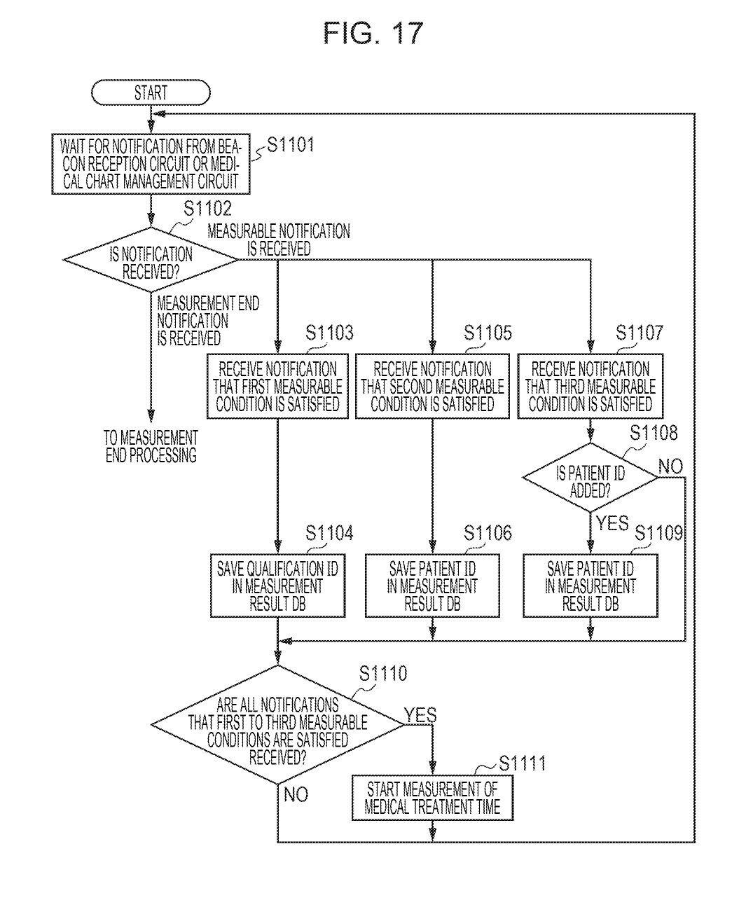

Specifically, the medical chart management device 410 monitors whether or not the electronic medical chart is in an access state (see S1001 in FIG. 16) and determines whether or not the access state to the electronic medical chart is changed (see S1002 in FIG. 16). If it is determined that the access state to the electronic medical chart is not changed, the medical chart management device 410 continuously monitors whether or not the electronic medical chart is in the access state. If it is determined that the access state to the electronic medical chart is changed, the medical chart management device 410 determines whether or not the electronic medical chart is newly accessed (see S1003 in FIG. 16). If it is determined that the electronic medical chart is not in a new access state, the medical chart management device 410 determines that the electronic medical chart is in a non-access state (see S1004 in FIG. 16). If it is determined that the electronic medical chart is in the non-access state, the medical chart management device 410 transmits a notification that the third measurable condition is no longer satisfied and the patient ID corresponding to the electronic medical chart in the non-access state to the measurement circuit 112 (see S1005 in FIG. 16). If it is determined that the electronic medical chart is in the new access state, the medical chart management device 410 transmits a notification that the third measurable condition is satisfied and a patient ID corresponding to the displayed electronic medical chart to the measurement circuit 112 (see S1006 of FIG. 16).

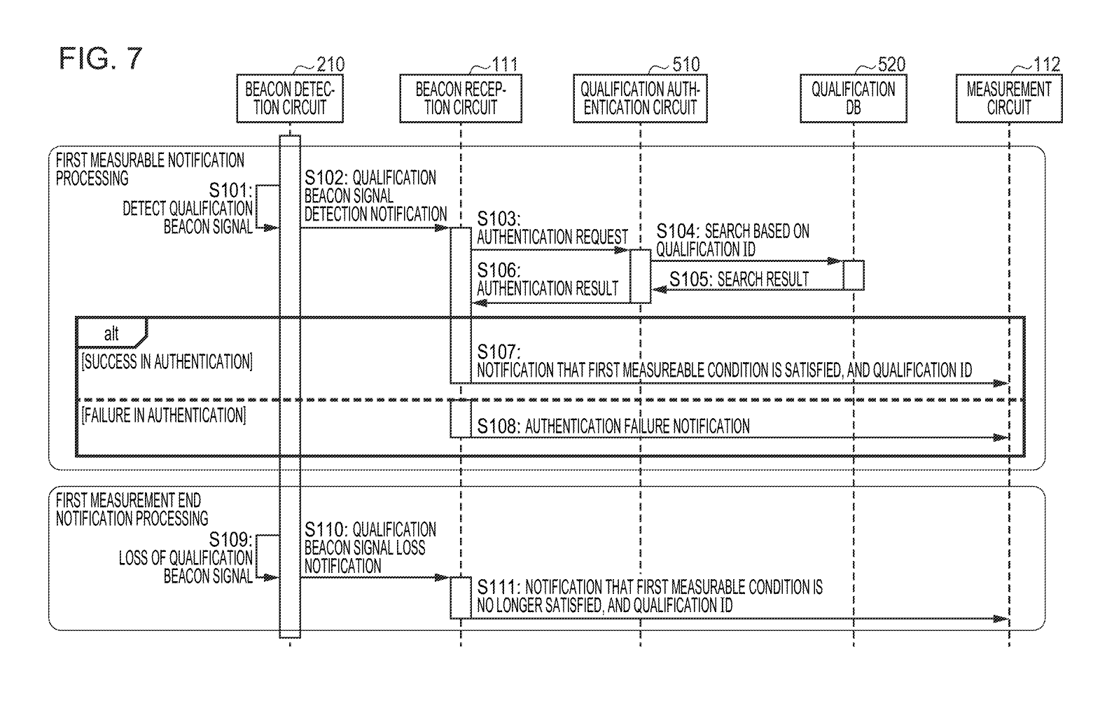

FIG. 7 is a sequence diagram illustrating an example of first measurable notification processing and first measurement end notification processing.

Here, the first measurable notification processing in which the beacon detection circuit 210 detects a qualification beacon signal and notifies the measurement circuit 112 that the first measurable condition is satisfied is first described with reference to the sequence diagram of FIG. 7. Next, the first measurement end notification processing in which the beacon detection circuit 210 does not detect the qualification beacon signal and notifies the measurement circuit 112 that the first measurable condition is not satisfied is described thereafter.

First, the first measurable notification processing of step S101 to step S108 is described.

In step S101, the beacon detection circuit 210 that monitors presence or absence of the qualification beacon signal transmitted by the qualification beacon 310 makes the processing proceed to S102, if the qualification beacon signal is detected.

The qualification beacon signal includes a qualification ID capable of identifying a work executor such as a doctor or a nurse carrying the qualification beacon 310.

In step S102, the beacon detection circuit 210 that detects the qualification beacon signal including the qualification ID transmits a qualification beacon signal detection notification to which the qualification ID is added to the beacon reception circuit 111, and makes the processing proceed to S103.

In step S103, the beacon reception circuit 111 that receives the qualification beacon signal detection notification to which the qualification ID is added transmits an authentication request based on the qualification ID to the qualification authentication circuit 510, and makes the processing proceed to S104.

In step S104, the qualification authentication circuit 510 that receives the authentication request based on the qualification ID performs search processing based on whether or not a corresponding qualification ID exists in the qualification information DB 520, and makes the processing proceed to S105.

In step S105, the qualification authentication circuit 510 that performs the search processing generates an authentication result notification including a search result of the qualification information DB 520, and makes the processing proceed to S106.

In the present embodiment, in a case where the qualification authentication circuit 510 that performs the search processing determines that the corresponding qualification ID exists in the qualification information DB 520, it is assumed that the authentication is successful. In addition, in a case where the qualification authentication circuit 510 that performs the search processing determines that the corresponding qualification ID does not exist in the qualification information DB 520, it is assumed that the authentication fails.

In step S106, the qualification authentication circuit 510 transmits the generated authentication result notification to the beacon reception circuit 111. If the authentication result notification is a notification to the effect that the authentication is successful, the qualification authentication circuit 510 makes the processing proceed to S107, and if the authentication result notification is a notification to the effect that the authentication fails, the qualification authentication circuit 510 makes the processing proceed to S108.

In step S107, the beacon reception circuit 111 that receives the authentication result notification to the effect that the authentication is successful transmits a notification that the first measurable condition is satisfied and the qualification ID to the measurement circuit 112. The measurement circuit 112 receives the notification that the first measurable condition is satisfied and the qualification ID from the beacon reception circuit 111, and ends the present processing.

In step S108, the beacon reception circuit 111 that receives the authentication result notification to the effect that the authentication fails transmits an authentication failure notification to the measurement circuit 112. The measurement circuit 112 receives the authentication failure notification from the beacon reception circuit 111, and ends the present processing.

Next, the first measurement end notification processing of step S109 to step S111 is described.

In step S109, the beacon detection circuit 210 that monitors presence or absence of the qualification beacon signal transmitted by the qualification beacon 310 makes the processing proceed to S110, if loss of the qualification beacon signal is detected.

In step S110, the beacon detection circuit 210 that detects the loss of the qualification beacon signal including the qualification ID transmits a qualification beacon signal loss notification to which the qualification ID is added to the beacon reception circuit 111, and makes the processing proceed to S111.

In step S111, the measurement circuit 112 that receives the qualification beacon signal loss notification transmits a notification that the first measurable condition is no longer satisfied, and a qualification ID corresponding to the lost qualification beacon signal to the measurement circuit 112. The measurement circuit 112 receives the notification that the first measurable condition is no longer satisfied and the qualification ID corresponding to the lost qualification beacon signal, and ends the present processing.

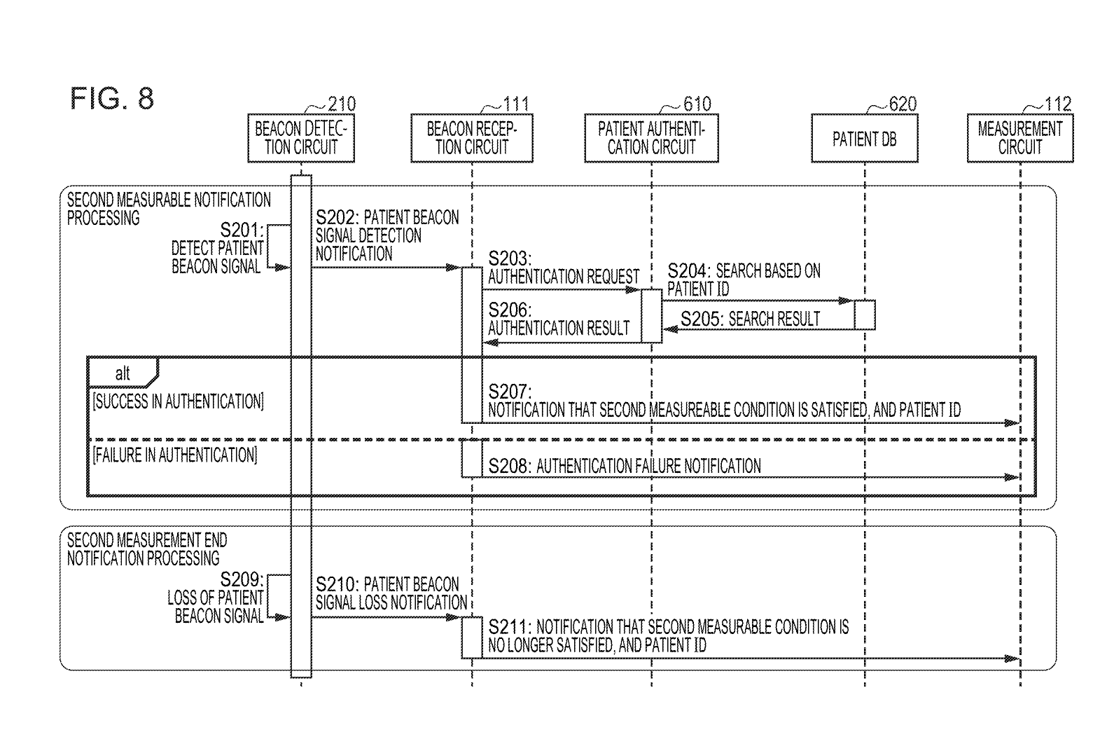

FIG. 8 is a sequence diagram illustrating an example of second measurable notification processing and second measurement end notification processing.

Here, the second measurable notification processing in which the beacon detection circuit 210 detects a patient beacon signal and notifies the measurement circuit 112 that the second measurable condition is satisfied is first described with reference to the sequence diagram of FIG. 8. Next, the second measurement end notification processing in which the beacon detection circuit 210 does not detect the patient beacon signal and notifies the measurement circuit 112 that the second measurable condition is not satisfied is described thereafter.

First, the second measurable notification processing of step S201 to step S208 is described.

In step S201, the beacon detection circuit 210 that monitors presence or absence of a patient beacon signal transmitted by the patient beacon 320 makes the processing proceed to S202, if the patient beacon signal is detected.

The patient beacon signal includes a patient ID capable of identifying a work subject such as a patient carrying the patient beacon 320.

In step S202, the beacon detection circuit 210 that detects the patient beacon signal including the patient ID transmits a patient beacon signal detection notification to which the patient ID is added to the beacon reception circuit 111, and makes the processing proceed to S203.

In step S203, the beacon reception circuit 111 that receives the patient beacon signal detection notification to which the patient ID is added transmits an authentication request based on the patient ID to the patient authentication circuit 610, and makes the processing proceed to S204.

In step S204, the patient authentication circuit 610 that receives the authentication request based on the patient ID performs search processing based on whether or not a corresponding patient ID exists in the patient information DB 620, and, and makes the processing makes the processing proceed to S205.

In step S205, the patient authentication circuit 610 that performs the search processing generates an authentication result notification including a search result of the patient information DB 620, and makes the processing proceed to S206.

In the present embodiment, in a case where the corresponding patient ID exists in the patient information DB 620, it is assumed that the authentication is successful. In addition, if the corresponding patient ID does not exist in the patient information DB 620, it is assumed that the authentication fails.

In step S206, the patient authentication circuit 610 transmits the generated authentication result notification to the beacon reception circuit 111. If the authentication result notification is a notification to the effect that the authentication is successful, the patient authentication circuit 610 proceeds to S207, and if the authentication result notification is a notification to the effect that the authentication fails, the patient authentication circuit 610 proceeds to S208.

In step S207, the beacon reception circuit 111 that receives the authentication result notification to the effect that the authentication is successful transmits a notification that the second measurable condition is satisfied and the patient ID to the measurement circuit 112. The measurement circuit 112 receives the notification that the second measurable condition is satisfied and the qualification ID from the beacon reception circuit 111, and ends the present processing.

In step S208, the beacon reception circuit 111 that receives the authentication result notification to the effect that the authentication fails transmits an authentication failure notification to the measurement circuit 112. The measurement circuit 112 receives the authentication failure notification from the beacon reception circuit 111, and ends the present processing.

Next, the second measurement end notification processing of step S209 to step S211 is described.

In step S209, the beacon detection circuit 210 that monitors presence or absence of the patient beacon signal transmitted by the patient beacon 320 makes the processing proceed to S210, if loss of the patient beacon signal is detected.

In step S210, the beacon detection circuit 210 that detects the loss of the patient beacon signal including the patient ID transmits a patient beacon signal loss notification to which the patient ID is added to the beacon reception circuit 111, and makes the processing proceed to S211.

In step S211, the beacon reception circuit 111 that receives the patient beacon signal loss notification transmits a notification that the second measurable condition is no longer satisfied, and a patient ID corresponding to the lost patient beacon signal to the measurement circuit 112. The measurement circuit 112 receives the notification that the second measurable condition is no longer satisfied and the patient ID corresponding to the lost patient beacon signal, and ends the present processing.

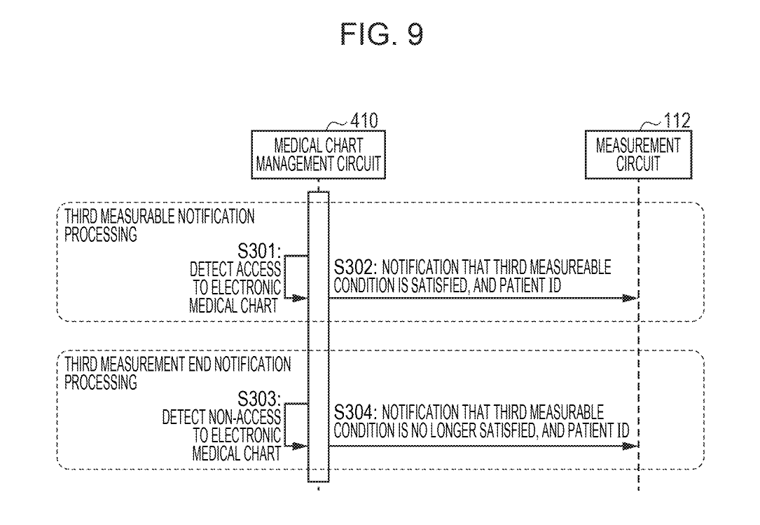

FIG. 9 is a sequence diagram illustrating an example of third measurable notification processing and third measurement end notification processing.

Here, the third measurable notification processing in which the medical chart management device 410 detects that the electronic medical chart is in an access state and notifies the measurement circuit 112 that a third measurable condition is satisfied is first described with reference to the sequence diagram of FIG. 9. Next, the third measurement end notification processing in which the medical chart management device 410 detects that the electronic medical chart is in a non-access state and notifies the measurement circuit 112 that the third measurable condition is not satisfied is described thereafter.

First, the third measurable notification processing of step S301 to step S302 is described.

In step S301, the medical chart management device 410 that monitors whether or not the electronic medical chart is in an access state makes the processing proceed to S302, if it is detected that the electronic medical chart is in the access state.

In step S302, the medical chart management device 410 transmits a notification that a third measurable condition is satisfied and a patient ID corresponding to the displayed electronic medical chart to the measurement circuit 112. The measurement circuit 112 receives the notification that the third measurable condition is satisfied and the patient ID corresponding to the displayed electronic medical chart, and ends the present processing.

Next, the third measurement end notification processing in step S303 to step S304 is described.

In step S303, when the medical chart management device 410 that monitors whether or not the electronic medical chart is displayed makes the processing proceed to S304, if it is detected that the electronic medical chart is in a non-access state.

In step S304, the medical chart management device 410 transmits a notification that the third measurable condition is no longer satisfied and a patient ID corresponding to the electronic medical chart in the non-access state to the measurement circuit 112. The measurement circuit 112 receives the notification that the third measurable condition is not satisfied and a patient ID corresponding to the electronic medical chart in the non-access state, and ends the present processing.

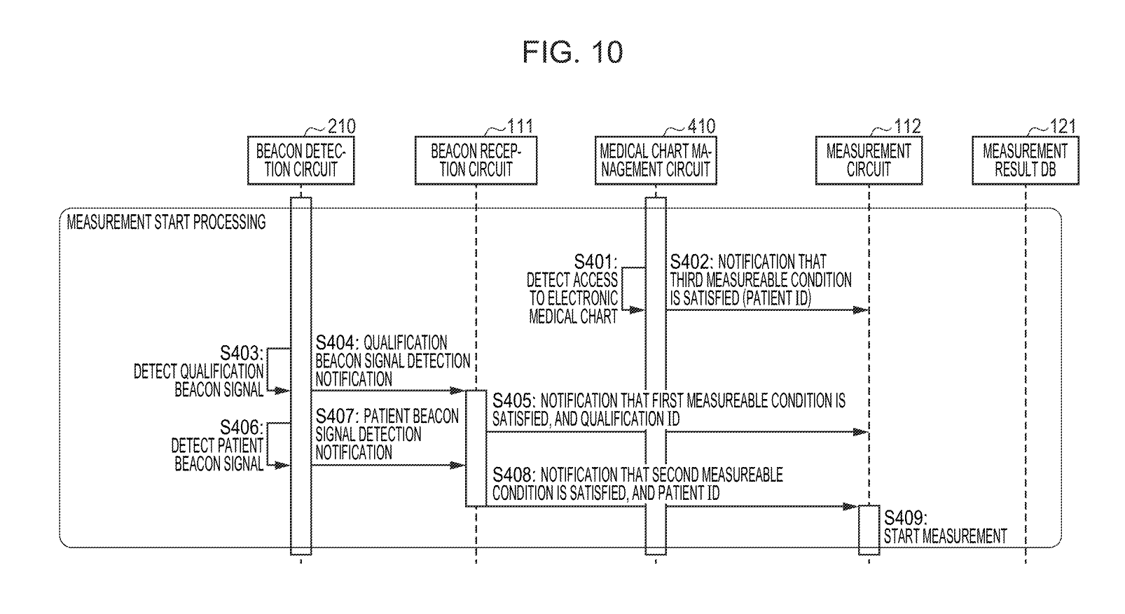

FIG. 10 is a sequence diagram illustrating an example of measurement start processing.

Here, a flow of starting measurement of medical treatment time when all the measurable conditions from the first measurable condition to the third measurable condition are satisfied is described with reference to the sequence diagram of FIG. 10.

In the present embodiment, it is described that the measurement circuit 112 receives notifications in the order of the notification that the third measurable condition is satisfied, the notification that the first measurable condition is satisfied, and the notification that the second measurable condition is satisfied, and the embodiment is not limited to this, and any order may be used.

First, measurement start processing in step S401 to step S409 is described.

In step S401 (corresponding to S301), the medical chart management device 410 that monitors whether or not the electronic medical chart is displayed makes the processing proceed to S402, if it is detected that the electronic medical chart is displayed.

In step S402 (corresponding to S302), the medical chart management device 410 transmits the notification that the third measurable condition is satisfied and a patient ID corresponding to the displayed electronic medical chart to the measurement circuit 112. The measurement circuit 112 receives the notification that the third measurable condition is satisfied and the patient ID corresponding to the displayed electronic medical chart, and makes the processing proceed to S403.

In step S403 (corresponding to S101), the beacon detection circuit 210 that monitors presence or absence of the qualification beacon signal transmitted by the qualification beacon 310 makes the processing proceed to S404, if the qualification beacon signal is detected.

In step S404 (corresponding to S102), the beacon detection circuit 210 that detects the qualification beacon signal including the qualification ID transmits the qualification beacon signal detection notification to which the qualification ID is added to the beacon reception circuit 111, and makes the processing proceed to S405.

In step S405 (corresponding to S107), the beacon reception circuit 111 that receives an authentication result notification to the effect that authentication is successful transmits a notification that a first measurable condition is satisfied and a qualification ID to the measurement circuit 112. The measurement circuit 112 receives the notification that the first measurable condition is satisfied and the qualification ID from the beacon reception circuit 111, and makes the processing proceed to S406.

In step S406 (corresponding to S201), the beacon detection circuit 210 that monitors presence or absence of a patient beacon signal transmitted by the patient beacon 320 makes the processing proceed to S407, if the patient beacon signal is detected.

In step S407 (corresponding to S202), the beacon detection circuit 210 that detects the patient beacon signal including the patient ID transmits a patient beacon signal detection notification to which the patient ID is added to the beacon reception circuit 111, and makes the processing proceed to S408.

In step S408 (corresponding to S207), the beacon reception circuit 111 that receives an authentication result notification that the authentication is successful transmits a notification that a second measurable condition is satisfied and a patient ID to the measurement circuit 112. The measurement circuit 112 receives the notification that the second measurable condition is satisfied and the qualification ID from the beacon reception circuit 111, and makes the processing proceed to S409.

In step S409, all the measurable conditions from the first measurable condition to the third measurable condition are satisfied, and thereby, the measurement circuit 112 starts measurement of the medical treatment time and ends the present processing.

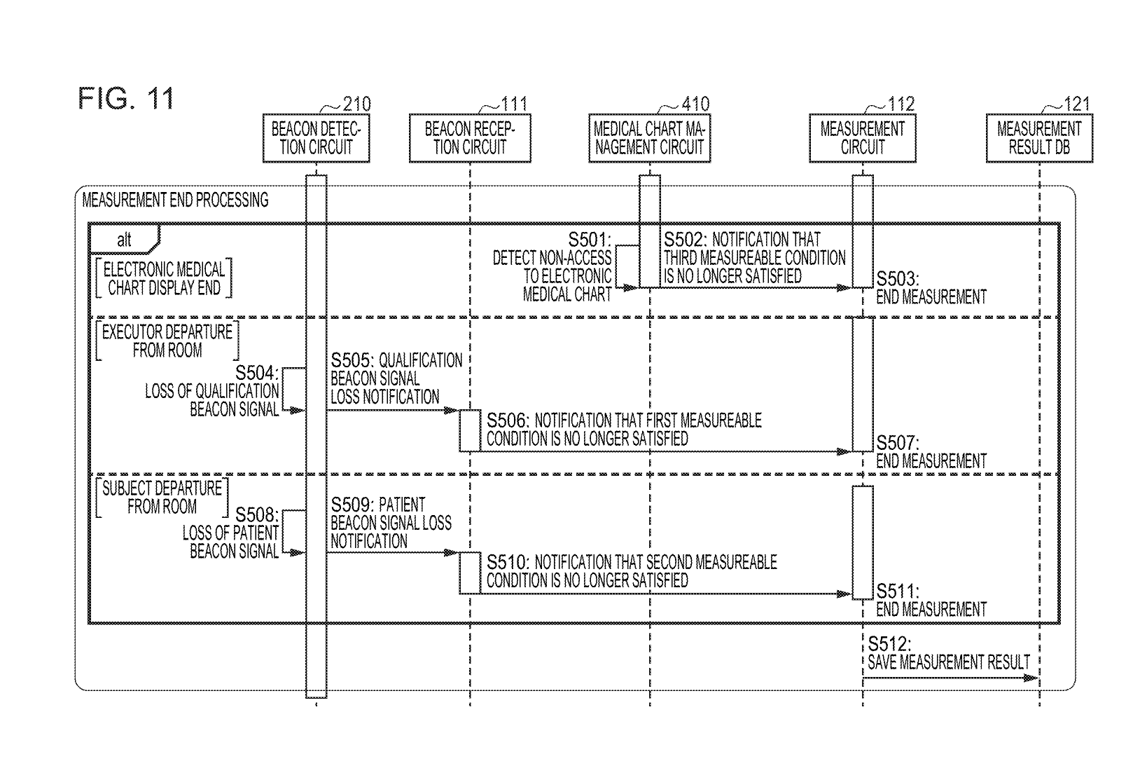

FIG. 11 is a sequence diagram illustrating an example of measurement end processing.

Here, a flow of ending measurement of medical treatment time when any one of the measurable conditions from the first measurable condition to the third measurable condition is no longer satisfied is described with reference to the sequence diagram of FIG. 11.

First, in step S501 to step S503, measurement end processing in a case where it is detected that the electronic medical chart is in a non-access state is described.

In step S501 (corresponding to S303), the medical chart management device 410 that monitors whether or not the electronic medical chart is displayed makes the processing proceed to S502, if it is detected that the electronic medical chart is in the non-access state.

In step S502 (corresponding to S304), the medical chart management device 410 transmits a notification that a third measurable condition is satisfied and a patient ID corresponding to an electronic medical chart in a non-access state to the measurement circuit 112. The measurement circuit 112 receives the notification that the third measurable condition is not satisfied and the patient ID corresponding to the electronic medical chart in the non-access state, and makes the processing proceed to S503.

In step S503, the measurement circuit 112 ends the measurement of the medical treatment time and makes the processing proceed to S512.

Next, in step S504 to step S507, measurement end processing in a case where loss of a qualification beacon signal is detected is described.

In step S504 (corresponding to S109), the beacon detection circuit 210 that monitors presence or absence of the qualification beacon signal transmitted by the qualification beacon 310 makes the processing proceed to S505, if the loss of the qualification beacon signal is detected.

In step S505 (corresponding to S110), the beacon detection circuit 210 that detects the loss of the qualification beacon signal including the qualification ID transmits a qualification beacon signal loss notification to which the qualification ID is added to the beacon reception circuit 111, and makes the processing proceed to S506.

In step S506 (corresponding to S111), the measurement circuit 112 that receives the qualification beacon signal loss notification transmits a notification that a first measurable condition is no longer satisfied and a qualification ID corresponding to the lost qualification beacon signal to the measurement circuit 112. The measurement circuit 112 receives the notification that the first measurable condition is no longer satisfied and the qualification ID corresponding to the lost qualification beacon signal, and makes the processing proceed to S507.

In step S507, the measurement circuit 112 ends the measurement of the medical treatment time and makes the processing proceed to S512.

Next, in step S508 to step S511, measurement end processing in a case where loss of a patient beacon signal is detected is described.

In step S508 (corresponding to S209), the beacon detection circuit 210 that monitors presence or absence of the patient beacon signal transmitted by the patient beacon 320 makes the processing proceed to S509, if the loss of the patient beacon signal is detected.

In step S509 (corresponding to S210), the beacon detection circuit 210 that detects the loss of the patient beacon signal including the patient ID transmits a patient beacon signal loss notification to which the patient ID is added to the beacon reception circuit 111, and makes the processing proceed to S510.

In step S510 (corresponding to S211), the beacon reception circuit 111 that receives the patient beacon signal loss notification transmits a notification that a second measurable condition is no longer satisfied and a patient ID corresponding to the lost patient beacon signal to the measurement circuit 112. The measurement circuit 112 receives the notification that the second measurable condition is no longer satisfied and the patient ID corresponding to the lost patient beacon signal, and makes the processing proceed to S511.

In step S511, the measurement circuit 112 ends the measurement of the medical treatment time and makes the processing proceed to S512.

In step S512, the measurement circuit 112 saves a measurement result of the medical treatment time in the measurement result DB 121, and ends the present processing.

FIG. 12 is a flowchart illustrating an example of a flow of processing in which the beacon detection circuit 210 monitors each beacon signal and transmits various notifications to the beacon reception circuit 111.

Here, a flow of processing in which the beacon detection circuit 210 monitors a qualification beacon signal or a patient beacon signal and transmits a beacon signal detection notification or a beacon signal loss notification to the beacon reception circuit 111 with reference to the flowchart of FIG. 12.

In step S601, the beacon detection circuit 210 starts monitoring a state (detection or loss) of each beacon signal in the qualification beacon signal or the patient beacon signal, and makes the processing proceed to S602.

In step S602, the beacon detection circuit 210 that monitors a state of the beacon signal determines whether or not the state of the beacon signal is changed. If it is determined that the state of the beacon signal is not changed, the beacon detection circuit 210 returns to step S601 and continuously monitors the beacon signal. If it is determined that the state of the beacon signal is changed, the beacon detection circuit 210 makes the processing proceed to S603.

In step S603, the beacon detection circuit 210 which determines that the state of the beacon signal is changed determines whether or not a new beacon signal is detected. If it is determined that the new beacon signal is not detected, the beacon detection circuit 210 makes the processing proceed to S604. If it is determined that a new beacon signal is detected, the beacon detection circuit 210 makes the processing proceed to S606.

In step S604 (corresponding to S109 or S209), the beacon detection circuit 210 which determines that a new beacon signal is not detected determines that the detected beacon signal is lost, and makes the processing proceed to S605.

In step S605 (corresponding to S110 or S210), the beacon detection circuit 210 which determines that the detected beacon signal is lost transmits a beacon signal loss notification to the beacon reception circuit 111, returns the processing to S601, and continuously monitors the beacon signal.

In step S606 (corresponding to S102 or S202), the beacon detection circuit 210 which determines that a new beacon signal is detected transmits a beacon signal detection notification to the beacon reception circuit 111, returns the processing to S601, and continuously monitor the beacon signal.

FIG. 13 is a flowchart illustrating an example of a flow of processing in which the beacon reception circuit 111 transmits various notifications and IDs to the measurement circuit 112, based on various notifications received from the beacon detection circuit 210.

Here, the flow of the processing in which the beacon reception circuit 111 transmits various notifications and IDs to the measurement circuit 112, based on various notifications received from the beacon detection circuit 210 is described with reference to the flowchart of FIG. 13.

In step S701, the beacon reception circuit 111 waits for a notification from the beacon detection circuit 210 and makes the processing proceed to S702.

In step S702, the beacon reception circuit 111 determines whether or not a notification is received from the beacon detection circuit 210. If it is determined that the beacon signal loss notification is received from the beacon detection circuit 210, the beacon reception circuit 111 makes the processing proceed to S703. If it is determined that the beacon signal detection notification is received from the beacon detection circuit 210, the beacon reception circuit 111 makes the processing proceed to S704.

In step S703, the beacon reception circuit 111 transmits a notification that the measurable condition is no longer satisfied and an ID and returns the processing proceed to step S701.

In step S704, the beacon reception circuit 111 determines whether or not the received ID exists in the patient information DB. If it is determined that the received ID exists in the patient information DB, the beacon reception circuit 111 makes the processing to S709. If it is determined that the received ID does not exist in the patient information DB, the beacon reception circuit 111 makes the processing proceed to S705.

In step S705, the beacon reception circuit 111 transmits an authentication request to the qualification authentication circuit 510 and makes the processing proceed to S706.

In step S706, the beacon reception circuit 111 receives the authentication result from the qualification authentication circuit 510 and makes the processing proceed to S707.

In step S707, the beacon reception circuit 111 determines whether or not the authentication result is successful in authentication. If it is determined that the authentication result fails in authentication, the beacon reception circuit 111 returns the processing to S701. If it is determined that the authentication result is successful in authentication, the beacon reception circuit 111 makes the processing proceed to S708.

In step S708, the beacon reception circuit 111 adds a qualification ID to a notification that a first measurable condition is satisfied, transmits the notification to the measurement circuit 112, and returns the processing to S701.

In step S709, the beacon reception circuit 111 adds a patient ID to a notification that a second measurable condition is satisfied, transmits the notification to the measurement circuit 112, and returns the processing to S701.

FIG. 14 is a flowchart illustrating an example of a flow of processing in which the qualification authentication circuit 510 receives an authentication request from the beacon reception circuit 111 and transmits various notifications to the beacon reception circuit 111.

Here, the flow of the processing in which the qualification authentication circuit 510 receives the authentication request from the beacon reception circuit 111 and transmits various notifications to the beacon reception circuit 111 is described with reference to the flowchart of FIG. 14.

In step S801 (corresponding to S103), the qualification authentication circuit 510 receives an authentication request based on the qualification ID from the beacon reception circuit 111 and makes the processing proceed to S802.

In step S802 (corresponding to S104), the qualification authentication circuit 510 which receives the authentication request based on the qualification ID performs search processing, based on whether or not a corresponding qualification ID exists in the qualification information DB 520 and makes the processing proceed to S803.

In step S803 (corresponding to S105), if it is determined that the corresponding qualification ID exists in the qualification information DB 520, the qualification authentication circuit 510 that performs the search processing makes the processing proceed to S804. If it is determined that the corresponding qualification ID does not exist in the qualification information DB 520, the qualification authentication circuit 510 makes the processing proceed to S805.

In step S804 (corresponding to S106), the qualification authentication circuit 510 transmits an authentication result notification to the effect that the authentication is successful to the beacon reception circuit 111 and ends the present processing.

In step S805 (corresponding to S106), the qualification authentication circuit 510 transmits an authentication result notification that the authentication fails to the beacon reception circuit 111, and ends the present processing.

FIG. 15 is a flowchart illustrating an example of a flow of processing in which the patient authentication circuit 610 receives an authentication request from the beacon reception circuit 111 and transmits various notifications to the beacon reception circuit 111.

Here, the flow of the processing in which the patient authentication circuit 610 receives the authentication request from the beacon reception circuit 111 and transmits various notifications to the beacon reception circuit 111 is described with reference to the flowchart of FIG. 15.

In step S901 (corresponding to S203), the patient authentication circuit 610 receives an authentication request based on a patient ID from the beacon reception circuit 111, and makes the processing proceed to S902.

In step S902 (corresponding to S204), the patient authentication circuit 610 which receives the authentication request based on the patient ID performs search processing, based on whether or not a corresponding patient ID exists in the patient information DB 620, and makes the processing proceed to S903.

In step S903 (corresponding to S205), the patient authentication circuit 610 that performs the search processing makes the processing proceed to S904, if the corresponding patient ID exists in the patient information DB 620. If it is determined that the corresponding patient ID does not exist in the patient information DB 620, the patient authentication circuit 610 makes the processing proceed to S905.

In step S904 (corresponding to S206), the patient authentication circuit 610 transmits an authentication result notification to the effect that authentication is successful to the beacon reception circuit 111 and ends the present processing.

In step S905 (corresponding to S206), the patient authentication circuit 610 transmits an authentication result notification that the authentication fails to the beacon reception circuit 111 and ends the present processing.

FIG. 16 is a flowchart illustrating an example of a flow of processing in which the medical chart management device 410 monitors whether or not the electronic medical chart is in an access state and transmits various notifications to the beacon reception circuit 111.

Here, the flow of the processing in which the medical chart management device 410 monitors whether or not the electronic medical chart is in an access state and transmits various notifications to the beacon reception circuit 111 is described with reference to the flowchart of FIG. 16.

In step S1001, the medical chart management device 410 starts monitoring whether or not the electronic medical chart is in an access state and makes the processing proceed to S1002.

In step S1002, the medical chart management device 410 that monitors whether or not the electronic medical chart is in an access state determines whether or not an access state to the electronic medical chart is changed. If it is determined that the access state to the electronic medical chart is not changed, the medical chart management device 410 returns the processing to S1001 and continuously monitors whether or not the electronic medical chart is in an access state. If it is determined that the access state to the electronic medical chart is changed, the medical chart management device 410 makes the processing proceed to S1003.

In step S1003, the medical chart management device 410, which determines that the access state to the electronic medical chart is changed, determines whether or not the electronic medical chart is in a new access state. If it is determined that the electronic medical chart is in a new access state, the medical chart management device 410 makes the processing proceed to S1004. If it is determined that the electronic medical chart is not in the new access state, the medical chart management device 410 makes the processing proceed to S1006.

In step S1004, the medical chart management device 410 which determines that the electronic medical chart is not in the new access state determines that the electronic medical chart is in a non-access state and makes the processing proceed to S1005.

In step S1005 (corresponding to step S304), the medical chart management device 410 which determines that the electronic medical chart is in the non-access state transmits a notification that a third measurable condition is no longer satisfied and the patient ID corresponding to the electronic medical chart in the non-access state to the measurement circuit 112, and returns the processing to S1001.

In step S1006 (corresponding to S302), the medical chart management device 410 which determines that the electronic medical chart is in a new access state transmits a notification that a third measurable condition is satisfied and a patient ID corresponding to the displayed electronic medical chart to the measurement circuit 112 and returns the processing to S1001.

FIG. 17 is a flowchart illustrating an example of a flow of measurement start processing in which the measurement circuit 112 starts measurement of medical treatment time.

Here, the flow of the measurement start processing in which the measurement circuit 112 starts measurement of the medical treatment time is described with reference to the flowchart of FIG. 17.

In step S1101, the measurement circuit 112 waits for a notification from the beacon reception circuit 111 or the medical chart management device 410, and makes the processing proceed to S1102.

In step S1102, the measurement circuit 112 determines whether a notification is received from the beacon reception circuit 111 or the medical chart management device 410. If it is determined that a measurable notification is received from the beacon reception circuit 111 or the medical chart management device 410, the measurement circuit 112 makes the processing proceed to S1103. If it is determined that a measurement end notification is received from the beacon reception circuit 111 or the medical chart management device 410, the measurement circuit 112 makes the processing proceed to the measurement end processing which is described below.

In step S1103, if it is determined that the measurable notification is a notification that a first measurable condition is satisfied and the qualification ID, the measurement circuit 112 makes the processing proceed to S1104.

In step S1104, the measurement circuit 112 saves the qualification ID in the measurement result DB and makes the processing proceed to S1110.

In step S1105, if it is determined that the measurable notification is a notification that a second measurable condition is satisfied and the patient ID, the measurement circuit 112 makes the processing proceed to S1106.

In step S1106, the measurement circuit 112 saves the patient ID in the measurement result DB 121 and makes the processing proceed to S1110.

In step S1107, if it is determined that the measurable notification is a notification that a third measurable condition is satisfied, the measurement circuit 112 makes the processing proceed to S1108.

In step S1108, the measurement circuit 112 determines whether or not the patient ID is added to the notification that the third measurable condition is satisfied. If it is determined that the patient ID is added to the notification that the third measurable condition is satisfied, the measurement circuit 112 makes the processing proceed to S1109. If it is determined that the patient ID is not added to the notification that the third measurable condition is satisfied, the measurement circuit 112 makes the processing proceed to S1110.

In step S1109, the measurement circuit 112 saves the patient ID in the measurement result DB 121 and makes the processing proceed to S1110.

In step S1110, the measurement circuit 112 determines whether or not the notifications that all the measurable conditions from the first measurable condition to the third measurable condition are satisfied are received. If it is determined that the notifications that all the measurable conditions from the first measurable condition to the third measurable condition are satisfied are received, the measurement circuit 112 makes the processing proceed to S1111. If it is determined that the notifications that all the measurable conditions from the first measurable condition to the third measurable condition are satisfied are not received, the measurement circuit 112 returns the processing to S1101.

In step S1110, the measurement circuit 112, which determines that the notifications that all the measurable conditions from the first measurable condition to the third measurable condition are satisfied are received, starts the measurement of the medical treatment time and returns the processing to S1101.

FIG. 18 is a flowchart illustrating an example of a flow of measurement end processing in which the measurement circuit 112 ends the measurement of the medical treatment time.

Here, the flow of the measurement end processing in which the measurement circuit 112 ends the measurement of the medical treatment time is described with reference to the flowchart of FIG. 18.

In step S1201, the measurement circuit 112 waits for a notification from the beacon reception circuit 111 or the medical chart management device 410 and makes the processing proceed to S1202.

In step S1202, the measurement circuit 112 determines whether or not a notification is received from the beacon reception circuit 111 or the medical chart management device 410. If it is determined that the measurable notification is received from the beacon reception circuit 111 or the medical chart management device 410, the measurement circuit 112 proceeds to the measurement start processing described above. If it is determined that the measurement end notification is received from the beacon reception circuit 111 or the medical chart management device 410, the measurement circuit 112 makes the processing proceed to S1203 to S1205, based on the type of the measurement end notification.

In step S1203, if it is determined that the measurement end notification is a notification that the first measurable condition is no longer satisfied and the qualification ID, the measurement circuit 112 makes the processing proceed to S1206.

In step S1204, if it is determined that the measurement end notification is a notification that the second measurable condition is no longer satisfied and the patient ID, the measurement circuit 112 makes the processing proceed to S1206.

In step S1205, if it is determined that the measurement end notification is a notification that the third measurable condition is no longer satisfied, the measurement circuit 112 makes the processing proceed to S1206.

In step S1206, the measurement circuit 112 ends the measurement of the medical treatment time and makes the processing proceed to S1207.

In step S1207, the measurement circuit 112 saves the qualification ID, the patient ID, and the measurement result in the measurement result DB 121, and returns the processing to S1201.

As described above, the management system starts the measurement of the medical treatment time, when a doctor, a nurse or the like and a patient exist and an electronic medical chart of the patient is displayed in a physical examination room and a treatment room, that is, when there is a high probability that a patient may be medically treated from a doctor, a nurse, or the like. In addition, the management system ends the measurement of the medical treatment time, when a work executor such as a doctor or a nurse or a patient as a work subject or any combination thereof does not exist in the physical examination room and the treatment room, or when the electronic medical chart of the patient is in a non-access state. In this way, since the management system may more accurately measure the medical treatment time, the doctor, the nurse, or the like who performs medical treatment may suggest more accurate waiting time to the patient. Thereby, the patient may confirm the waiting time suggested by the management system, the patient may more easily utilize the waiting time without waiting near the medical treatment room.

In addition, the management system monitors and detects display of a qualification beacon signal, a patient beacon signal, and the electronic medical chart, and thereby, it is possible to perform automatic measurement without measurement of medical treatment time performed by a doctor, a nurses or the like.

### Second Embodiment

In a second embodiment, a patient ID that may specify a patient is not given to a patient beacon signal.

Specifically, in a case where the electronic medical chart displayed by the display terminal 400 includes the patient ID, and the detection terminal 200 detects the patient beacon signal, provisional identification information (hereinafter, also referred to as “provisional ID”) is added to a notification that a second measurable condition is satisfied. The management server 100 associates the patient ID included in the electronic medical chart with the provisional ID.

Thereby, since the patient ID by which a patient may be specified from the patient beacon signal is not able to be read, the patient ID is not able to associate with personal information included in the electronic medical chart of the patient, and leakage of the personal information of the patient may be reduced.

For example, a universally unique identifier (UUID) or the like is used as the provisional ID.

In addition, in a case where the patient ID and the provisional ID are associated with each other, even if the display terminal 400 detects that the display of the electronic medical chart is ended and notifies that the third measurable condition is no longer satisfied, the management server 100 does not end the measurement of the medical treatment time. Thereby, it is considered when a doctor physically examines a patient in a physical examination room, then an inspection technician treats the patient in a treatment room, and the doctor physically examines the patient again in the physical examination room. In this case, if the doctor temporarily ends the display of the electronic medical chart in the physical examination room when the first physical examination is ended, the measurement of the medical treatment time is ended regardless of a subsequent treatment and physical examination. Accordingly, even if the display terminal 400 detects end of the display of the electronic medical chart and notifies that the third measurable condition is no longer satisfied, in a case where the detection terminal 200 continuously detects the patient beacon signal in the physical examination room or the treatment room, the information processing server does not end the measurement of the medical treatment time.

It is assumed that, when the patient moves between a physical examination room B and a treatment room C, the patient beacon signal is continuously detected by detection terminals 200a and 200b.

In the present embodiment, a mechanism called “physical examination” is used as a method of identifying a patient having a beacon to which a specific ID is not given.

As a sequence for a doctor to physically examine a patient, the doctor or a nurse first displays a mark on an electronic medical chart of a patient to be called, the doctor or the nurse calls the patient, and thereafter, the called patient enters a physical examination room. At this time, as a person who enters the physical examination room is exactly the patient of the displayed electronic medical chart and the provisional ID of the beacon owned by the patient is associated with the patient ID included in the information of the electronic medical chart, it is possible to identify the patient.

In a case of a bank, a government office, or the like, instead of a hospital, a customer or a resident who comes to a counter is a subject of receipt and payment information or resident information of the displayed account. Accordingly, the management system may identify the customer or the resident by associating the provisional ID of the beacon owned by the customer or the resident with an ID included in the receipt and payment information or the resident information.

A curing action is often performed without viewing the electronic medical chart as a method of measuring the treatment time, and there is often no display terminal that displays the electronic medical chart in a place where the curing action is performed. Accordingly, since there is often a case where a physical examination is performed before and after the curing action, it is possible to identify a patient to be measured at a stage of a physical examination action by using information on the patient identified at the time of a physical examination at the time of curing.

Next, in the second embodiment, in order to describe a configuration different from the configuration according to the first embodiment, description is made with reference to the following drawings.

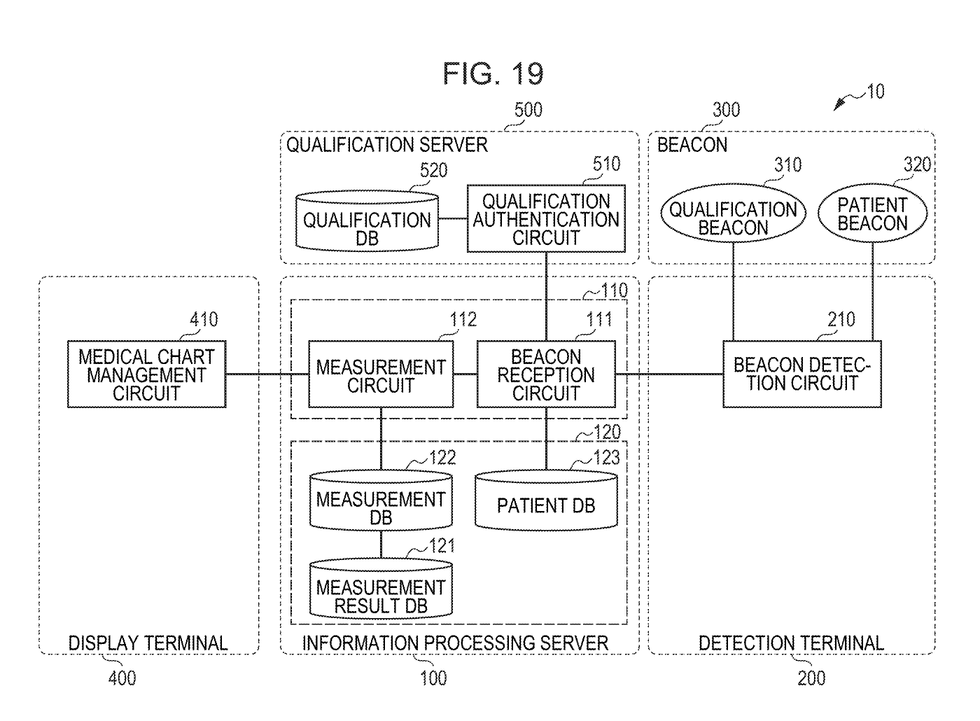

FIG. 19 is a block diagram illustrating an example of a functional configuration of a management system 10 according to the second embodiment. A point different from the functional configuration of the management system 10 according to the first embodiment illustrated in FIG. 1 is that there is no demand to authenticate a patient ID, and thus, the patient information DB is incorporated into the management server 100 without the patient server 600 including the patient authentication circuit 610.

Hereinafter, a sequence diagram and a flowchart different from those according to the first embodiment are described in the second embodiment.

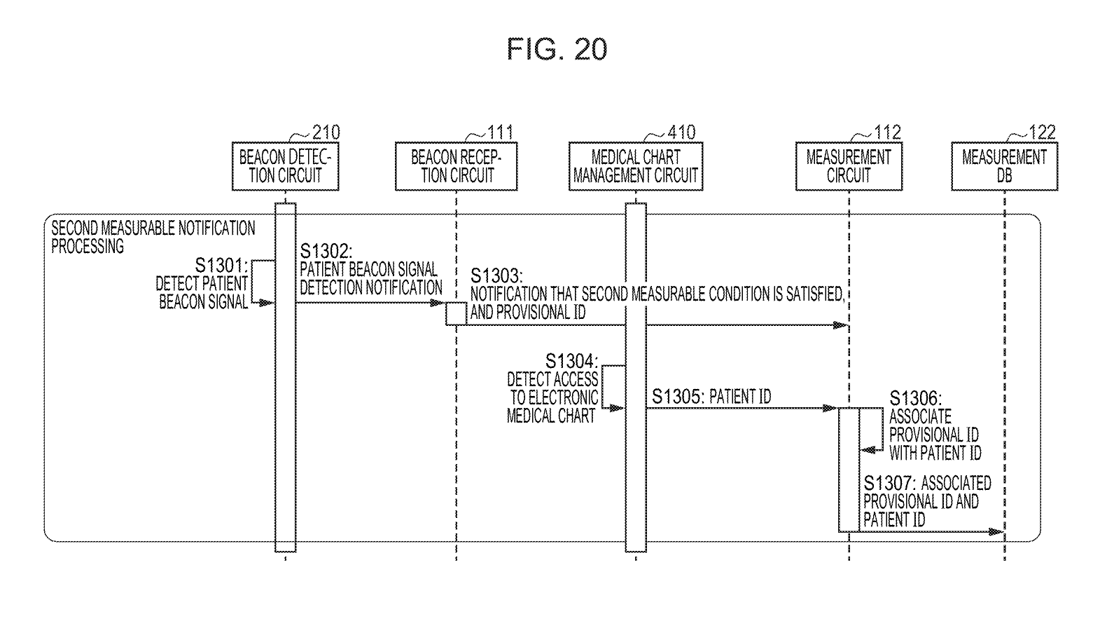

FIG. 20 is a sequence diagram illustrating an example of second measurable notification processing and second measurement end notification processing according to the second embodiment.

Here, the second measurable notification processing in which the beacon detection circuit 210 detects a patient beacon signal including a provisional ID and notifies the measurement circuit 112 that a second measurable condition is satisfied is first described with reference to the sequence diagram of FIG. 20.

In step S1301, if the patient beacon signal is detected, the beacon detection circuit 210 that monitors presence or absence of the patient beacon signal transmitted by the patient beacon 320, makes the processing proceed to S1302.

Unlike the first embodiment, the patient beacon signal does not include a patient ID.

In step S1302, the beacon detection circuit 210 that detects the patient beacon signal transmits a patient beacon signal detection notification to the beacon reception circuit 111 and makes the processing proceed to S1303.

In step S1303, the beacon reception circuit 111 that receives the patient beacon signal detection notification adds a provisional ID to a notification that a second measurable condition is satisfied, transmits the notification to the measurement circuit 112, and makes the processing proceed to S1304.

In step S1304, the medical chart management device 410 that monitors whether or not the electronic medical chart is in an access state makes the processing proceed to S1305, if it is detected that the electronic medical chart is in the access state.

In step S1305, the medical chart management device 410, which detects that the electronic medical chart is in the access state, transmits a notification that a third measurable condition is satisfied and a patient ID corresponding to the displayed electronic medical chart to the measurement circuit 112.

In step S1306, the measurement circuit 112 receives the notification that the third measurable condition is satisfied and the patient ID corresponding to the displayed electronic medical chart, associates the provisional ID received in step S1303 with the patient ID received in the present processing, and makes the processing proceed to S1307.

In step S1307, the measurement circuit 112 transmits the provisional ID and the patient ID associated with each other to the measurement DB 122 to save therein.

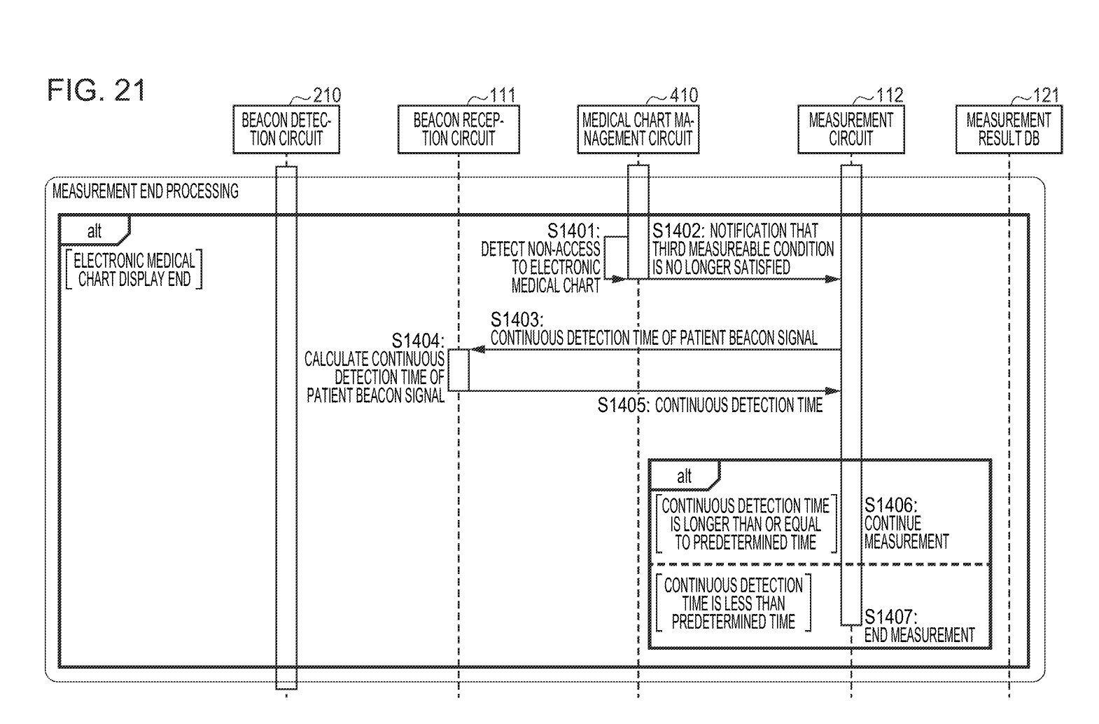

FIG. 21 is a sequence diagram illustrating an example of a part of measurement end processing according to the second embodiment.

Here, a flow of ending measurement of medical treatment time when a third measurable condition is no longer satisfied is described with reference to the sequence diagram of FIG. 21.

In step S1401, the medical chart management device 410 that monitors whether or not an electronic medical chart is accessed makes the processing proceed to S1402, if the electronic medical chart is in a non-access state.

In step S1402, the medical chart management device 410 transmits a notification that a third measurable condition is no longer satisfied to the measurement circuit 112. The measurement circuit 112 receives the notification that the third measurable condition is not satisfied, and makes the processing proceed to S1403.

In step S1403, the measurement circuit 112 transmits an output request of continuous detection time of z patient beacon signal to the beacon reception circuit 111, and makes the processing proceed to S1404.

In step S1404, the beacon reception circuit 111 calculates the continuous detection time of the patient beacon signal, and makes the processing proceed to S1405.

In step S1405, the beacon reception circuit 111 transmits the calculated continuous detection time to the measurement circuit 112. The measurement circuit 112 receives the continuous detection time and makes the processing proceed to S1406.

In step S1406, if the measurement circuit 112 determines that the received continuous detection time is longer than or equal to a predetermined time, the measurement circuit 112 continuously measures the medical treatment time and ends the present processing. If it is determined that the continuous detection time is no longer than the predetermined time, the measurement circuit 112 makes the processing proceed to S1407.

In step S1407, since the received continuous detection time is shorter than the predetermined time, the measurement circuit 112 ends the measurement of the medical treatment time and ends the present processing.

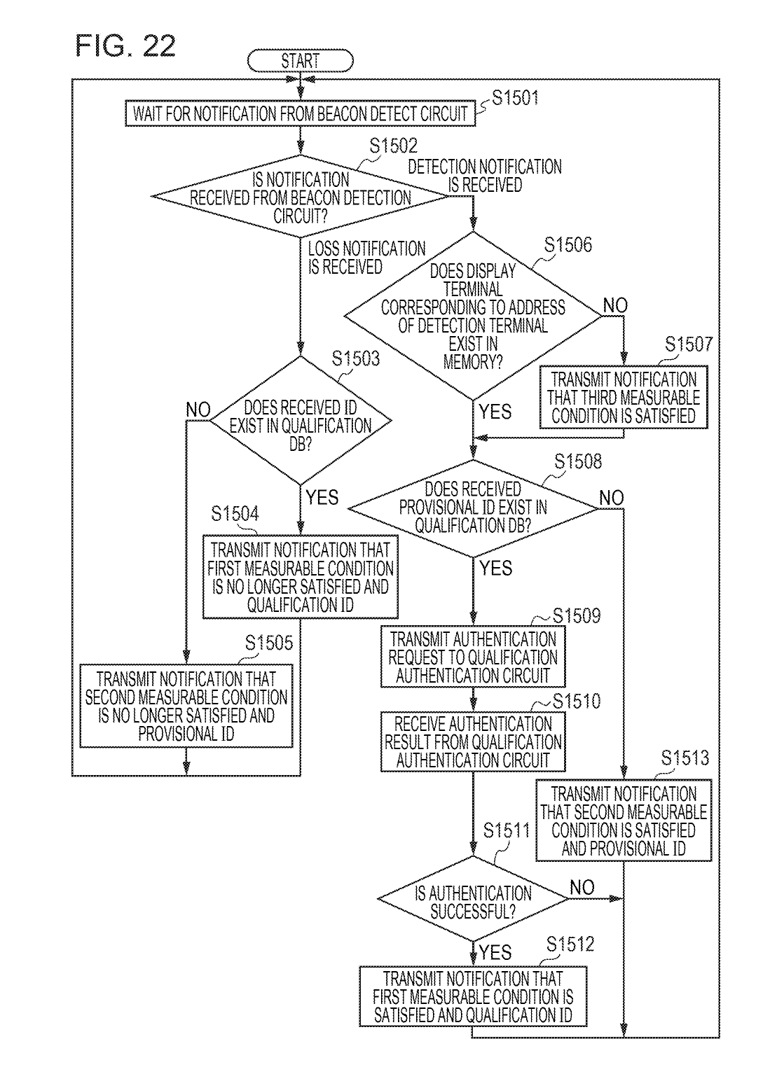

FIG. 22 is a sequence diagram illustrating an example of a second measurable notification processing and a second measurement end notification processing according to the second embodiment.

Here, the second measurable notification processing in which the beacon detection circuit 210 detects a patient beacon signal including a provisional ID and notifies the measurement circuit 112 that a second measurable condition is satisfied is described with reference to the sequence diagram of FIG. 22.

In step S1501, the beacon reception circuit 111 waits for notification from the beacon detection circuit 210 and makes the processing proceed to S1502.

In step S1502, the beacon reception circuit 111 determines whether or not a notification is received from the beacon detection circuit 210. If it is determined that a beacon signal loss notification is received from the beacon detection circuit 210, the beacon reception circuit 111 makes the processing proceed to S1503. If it is determined that the beacon signal detection notification is received from the beacon detection circuit 210, the beacon reception circuit 111 makes the processing proceed to S1506.

In step S1503, the beacon reception circuit 111 determines whether or not the received ID exists in a qualification information DB. If it is determined that the received ID exists in the qualification information DB, the beacon reception circuit 111 makes the processing proceed to S1504. If it is determined that the received ID does not exist in the patient information DB, the beacon reception circuit 111 makes the processing proceed to S1505.

In step S1504, the beacon reception circuit 111 adds the qualification ID to a notification that a first measurable condition is no longer satisfied, transmits the notification to the measurement circuit 112, and returns the processing to step S1501.

In step S1505, the beacon reception circuit 111 adds a provisional ID to a notification that a second measurable condition is no longer satisfied, transmits the notification to the measurement circuit 112, and returns the processing to step S1501.

In step S1506, the beacon reception circuit 111 determines whether or not a display terminal corresponding to a MAC address of a detection terminal exists in a memory. If it is determined that the display terminal corresponding to the MAC address of the detection terminal exists in the memory, the beacon reception circuit 111 makes the processing proceed to S1508. If it is determined that the display terminal corresponding to the MAC address of the detection terminal does not exist in the memory, the beacon reception circuit 111 makes the processing proceed to S1507.

In step S1507, the beacon reception circuit 111 transmits a notification that a third measurable condition is satisfied to the measurement circuit 112 and makes the processing proceed to S1508.

In step S1508, the beacon reception circuit 111 determines whether or not the received provisional ID exists in a qualification information DB. The beacon reception circuit 111 which determines that the received provisional ID exists in the qualification information DB makes the processing proceed to S1509. The beacon reception circuit 111 which determines that the received provisional ID does not exist in the qualification information DB makes the processing proceed to S1513.

In step S1509, the beacon reception circuit 111 transmits an authentication request to the qualification authentication circuit 510 and makes the processing proceed to S1510.

In step S1510, the beacon reception circuit 111 receives an authentication result from the qualification authentication circuit 510 and makes the processing proceed to S1511.

In step S1511, the beacon reception circuit 111 determines whether or not the authentication result is successful in authentication. If it is determined that the authentication result fails in authentication, the beacon reception circuit 111 returns the processing to S1501. If it is determined that the authentication result is successful in authentication, the beacon reception circuit 111 makes the processing proceed to S1512.

In step S1512, the beacon reception circuit 111 transmits a notification that a first measurable condition is satisfied and a qualification ID to the measurement circuit 112 and returns the processing to S1501.

In step S1513, the beacon reception circuit 111 adds a provisional ID to a notification that a second measurable condition is satisfied, transmits the notification to the measurement circuit 112, and returns the processing to S1501.

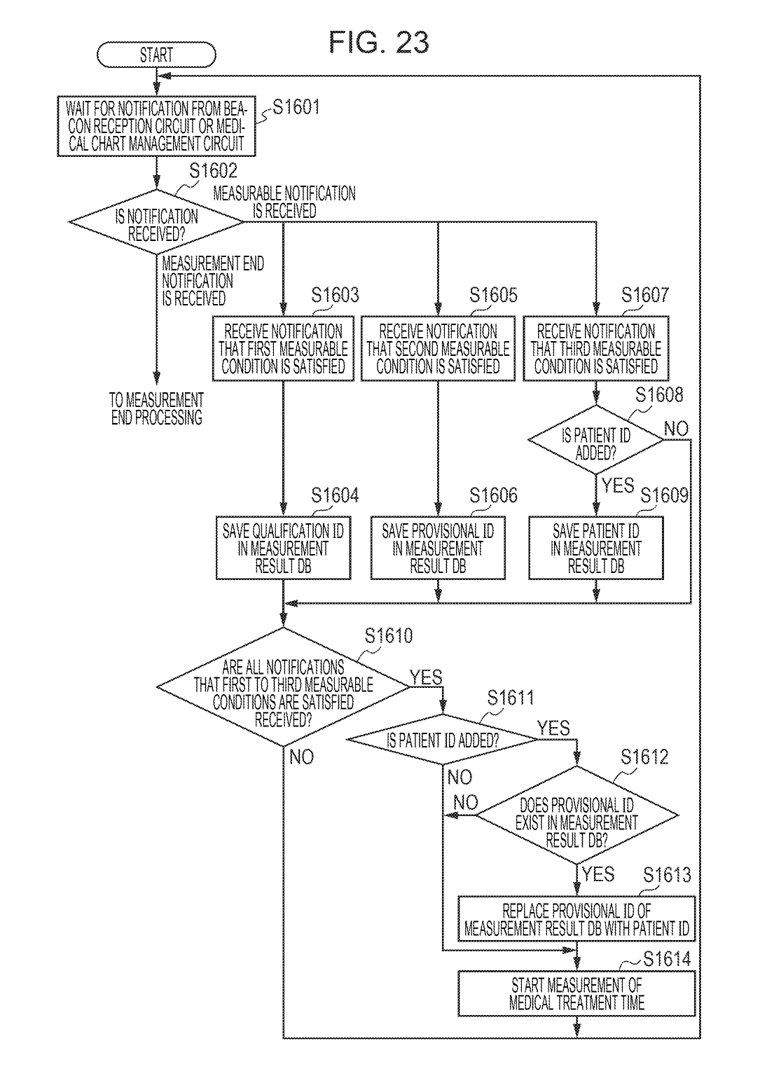

FIG. 23 is a flowchart illustrating an example of a flow of measurement end processing in which the measurement circuit 112 ends measurement of medical treatment time.

Here, the flow of the measurement end processing in which the measurement circuit 112 ends the measurement of the medical treatment time is described with reference to the flowchart of FIG. 23.

In step S1601, the measurement circuit 112 waits for a notification from the beacon reception circuit 111 or the medical chart management device 410, and makes the processing proceed to S1602.

In step S1602, the measurement circuit 112 determines whether or not a notification is received from the beacon reception circuit 111 or the medical chart management device 410. If it is determined that a measurable notification is received from the beacon reception circuit 111 or the medical chart management device 410, the measurement circuit 112 proceeds to the measurement start processing described above. If it is determined that a measurement end notification is received from the beacon reception circuit 111 or the medical chart management device 410, the measurement circuit 112 makes the processing proceed to S1603, S1605, and S1607 based on the type of the measurement end notification.

In step S1603, if it is determined that the measurement end notification is a notification that a first measurable condition is satisfied and a qualification ID, the measurement circuit 112 ends measurement of medical treatment time, and makes the processing proceed to S1604.

In step S1604, the measurement circuit 112 saves the qualification ID in the measurement result DB and makes the processing proceed to S1610.

In step S1605, if it is determined that the measurement end notification is a notification that a second measurable condition is satisfied and a patient ID, the measurement circuit 112 ends the measurement of the medical treatment time and makes the processing proceed to S1606.

In step S1606, the measurement circuit 112 saves the provisional ID in the measurement result DB and makes the processing proceed to S1610.

In step S1607, if it is determined that the measurement end notification is a notification that a third measurable condition is satisfied, the measurement circuit 112 makes the processing proceed to S1608.

In step S1608, the measurement circuit 112 determines whether or not the patient ID is added to a notification that a third measurable condition is satisfied. If it is determined that the patient ID is added to the notification that the third measurable condition is satisfied, the measurement circuit 112 makes the processing proceed to S1609. If it is determined that the patient ID is not added to the notification that the third measurable condition is satisfied, the measurement circuit 112 makes the processing proceed to S1610.

In step S1609, the measurement circuit 112 saves the patient ID in the measurement result DB 121 and makes the processing proceed to S1610.

In step S1610, the measurement circuit 112 determines whether or not notifications that all the measurable conditions from the first measurable condition to the third measurable condition are satisfied are received. If it is determined that the notifications that all the measurable conditions from the first measurable condition to the third measurable condition are satisfied are received, the measurement circuit 112 makes the processing proceed to S1611. If it is determined that the notifications that all the measurable conditions from the first measurable condition to the third measurable condition are satisfied are not received, the measurement circuit 112 returns the processing to S1601.

In step S1611, the measurement circuit 112 determines whether or not a patient ID is added to a notification that a third measurable condition is satisfied. If it is determined that the patient ID is added to the notification that the third measurable condition is satisfied, the measurement circuit 112 makes the processing proceed to S1612. If it is determined that the patient ID is not added to the notification that the third measurable condition is satisfied, the measurement circuit 112 makes the processing proceed to S1614.

In step S1612, the measurement circuit 112 determines whether or not a provisional ID exists in a measurement result DB. The measurement circuit 112 which determines that the provisional ID exists in the measurement result DB makes the processing proceed to S1613. The measurement circuit 112 which determines that the provisional ID does not exist in the measurement result DB makes the processing proceed to S1614.

In step S1613, the measurement circuit 112 replaces the provisional ID of the measurement result DB with the patient ID and makes the processing proceed to S1614.

In step S1614, the measurement circuit 112 starts the measurement of the medical treatment time and returns the processing to step S1601.

### Third Embodiment

In a third embodiment, the management system 10 more accurately measures the time demanded for a counter service which is introduced to a bank and is service time of the bank. Thereby, the bank may suggest more accurate waiting time to a customer, based on a measurement result, and thus, the customer does not have to wait near the counter and may effectively utilize the waiting time.

Here, in the present embodiment, the time demanded for the counter service is time until a service requested after being called at a counter is completed and the service is called again at the counter.

Information Processing System

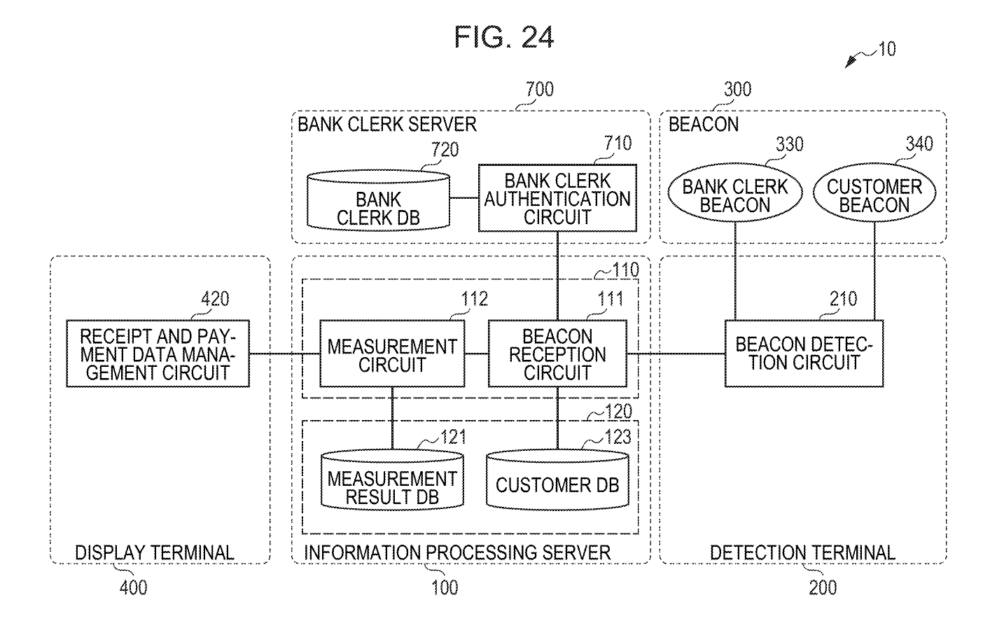

FIG. 24 is a block diagram illustrating an example of a functional configuration of a management system 10 according to a third embodiment.

As illustrated in FIG. 24, the management system 10 includes the management server 100, the detection terminal 200, a bank clerk beacon 330, a customer beacon 340, the display terminal 400, and a bank clerk server 700.

The management server 100 is communicably connected to the detection terminal 200, the display terminal 400, and the bank clerk server 700.

The detection terminal 200 may receive and detect a bank clerk beacon signal (executor existence signal) transmitted from a bank clerk beacon 330 carried by a bank clerk as a work executor in a counter or a waiting room. In the case of detecting the bank clerk beacon signal, the detection terminal 200 transmits a notification that a first measurable condition is satisfied to the management server 100.

In addition, the detection terminal 200 may receive and detect a customer beacon signal (subject existence signal) transmitted from the customer beacon 340 carried by a customer as a work subject, in a counter or a waiting room, in the same manner as the bank clerk beacon signal. In the case of detecting the customer beacon signal, the detection terminal 200 transmits a notification that a second measurable condition is satisfied to the management server 100.

In the case of detecting that information on receipt and payment data of an account of a customer is in an access state, the display terminal 400 transmits a notification that a third measurable condition is satisfied to the management server 100.

When all the measurable conditions from the first measurable condition to the third measurable condition are satisfied, the management server 100 starts measuring the time demanded for the counter service as the service time from the notification received from the detection terminal 200 and the display terminal 400. When any one of the measurable conditions is no longer satisfied, the management server 100 ends the measurement of the time demanded for the counter service.

When a bank clerk and a customer exist together at the counter or the waiting room and the receipt and payment data of the account of the customer is displayed, the management server 100 starts the measurement of the time demanded for the counter service. In addition, when a bank clerk as a work executor or a customer as a work subject or any combination thereof does not exist in the counter or the waiting room, or when the receipt and payment data of the account of the customer is in a non-access state, the management server 100 ends the measurement of the time demanded for the counter service.

The management server 100 transmits an authentication request to a bank clerk authentication circuit 710 included in the bank clerk server 700 and specifies a bank clerk who carries the bank clerk beacon 330, based on a bank clerk ID included in a bank clerk beacon signal.

The management server 100 transmits the authentication request and specifies a customer who carries the customer beacon 340, based on the customer ID included in the customer beacon signal.

As described above, the management system starts measurement of time demanded for a counter service, when a bank clerk and a customer exist in a counter or a waiting room and receipt and payment data of an account of the customer is displayed. In addition, the management system ends the measurement of the time demanded for the counter service, when the bank clerk as a work executor or the customer as a work subject or any combination thereof does not exist in the counter or the waiting room, or when the receipt and payment data of the account of the customer is in a non-access state. As such, the management system may more accurately measure the time demanded for the counter service, and thereby, a bank clerk may suggest more accurate waiting time to a customer. Thereby, by confirming the waiting time suggested by the management system, a customer easily and effectively use the waiting time without waiting near the counter.

In addition, the management system monitors and detects that the bank clerk beacon signal, the customer beacon signal, and the receipt and payment data of the account are displayed, and thereby, the time demanded for a bank clerk to perform the counter service may be automatically measured without performing the work for measuring the time.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing server comprising:
a reception circuit configured to transmit a notification that a first measurable condition is satisfied in a case where an executor existence signal indicating existence of a work executor is detected, a notification that a second measurable condition is satisfied in a case where a subject existence signal indicating existence of a work subject is detected, and a notification that a third measurable condition is satisfied in a case where an access state of the work subject to information is detected; and
a measurement circuit, coupled to the reception circuit, configured to start measurement of service time that is provided to the work subject when all measurable conditions from the first measurable condition to the third measurable condition are satisfied, and end the measurement of service time when any one of the measurable conditions is no longer satisfied.

2. The information processing server according to claim 1, the reception circuit initiates an authentication process to perform authentication of the executor existence signal and the subject existence signal prior to transmitting the notification that the first measurable condition is satisfied and the notification that the second measurable condition is satisfied.

3. The information processing server according to claim 2, wherein the authentication process includes comparing an identification value of the work executor to identification values stored in a first database and comparing an identification value of the work subject to identification values stored in a second database.

4. The information processing server according to claim 3, wherein the identification value of the work executor is included in the executor existence signal and the identification value of the work subject is included in the subject existence signal.

5. An information processing method causing a computer to execute a process comprising:
transmitting a notification that a first measurable condition is satisfied in a case where an executor existence signal indicating existence of a work executor is detected, a notification that a second measurable condition is satisfied in a case where a subject existence signal indicating existence of a work subject is detected, and a notification that a third measurable condition is satisfied in a case where an access state of the work subject to information is detected; and
starting measurement of service time that is provided to the work subject when all measurable conditions from the first measurable condition to the third measurable condition are satisfied, and ending the measurement of service time when any one of the measurable conditions is no longer satisfied.

6. The information processing method according to claim 5, further comprising:
initiating an authentication process to perform authentication of the executor existence signal and the subject existence signal prior to transmitting the notification that the first measurable condition is satisfied and the notification that the second measurable condition is satisfied.

7. The information processing method according to claim 6, wherein the authentication process includes comparing an identification value of the work executor to identification values stored in a first database and comparing an identification value of the work subject to identification values stored in a second database.

8. The information processing method according to claim 7, wherein the identification value of the work executor is included in the executor existence signal and the identification value of the work subject is included in the subject existence signal.

9. An information processing system comprising:
a detection terminal configured to provide a notification that a first measurable condition is satisfied in a case where an executor existence signal indicating existence of a work executor is detected, and a notification that a second measurable condition is satisfied in a case where a subject existence signal indicating existence of a work subject is detected;
a terminal configured to provide a notification that a third measurable condition is satisfied in a case where an access state of the work subject to information is detected; and
an information processing server configured to start measurement of service time that is provided to the work subject from notifications which are received from the detection terminal and the terminal, when all measurable conditions from the first measurable condition to the third measurable condition are satisfied, and configured to end the measurement of the service time when any one of the measurable conditions is no longer satisfied.

10. The information processing system according to claim 9,
wherein, when the terminal detects release of the access state of the work subject to information and provides a notification that the third measurable condition is no longer satisfied, in a case where the detection terminal continuously detects the subject existence signal in a predetermined region, the information processing server continues the measurement of the service time.

11. The information processing system according to claim 9, further comprising:
an authentication device configured to perform authentication of the executor existence signal and the subject existence signal prior to the detection terminal providing the notification that the first measurable condition is satisfied and the notification that the second measurable condition is satisfied.

12. The information processing system according to claim 11, wherein the authentication includes comparing an identification value of the work executor to identification values stored in a first database and comparing an identification value of the work subject to identification values stored in a second database.

13. The information processing system according to claim 12, wherein the identification value of the work executor is included in the executor existence signal and the identification value of the work subject is included in the subject existence signal.
